SEQUENCE LISTING (7 sequences)

SEQ ID NO: 1 - DNA, 26 bp, Artificial Sequence
  Primer
accagaatacgacttggagttgataa

SEQ ID NO: 2 - DNA, 20 bp, Artificial Sequence
  Primer
cacccttttgccagatgcat

SEQ ID NO: 3 - DNA, 17 bp, Artificial Sequence
  Primer
ctcgacgttgtcactga

SEQ ID NO: 4 - DNA, 16 bp, Artificial Sequence
  Primer
ccatccgagtacgtgc

SEQ ID NO: 5 - DNA, 19 bp, Artificial Sequence
  Primer
gaaggtgaaggtcggagtc

SEQ ID NO: 6 - DNA, 20 bp, Artificial Sequence
  Primer
gaagatggtgatgggatttc

SEQ ID NO: 7 - DNA, 23 bp, Artificial Sequence
  Primer
gatggcctattggcctggagggg

(19) United States
(12) Patent Application Publication
(10) Pub. No.: US 2004/0229936 A1
(43) Pub. Date: Nov. 18, 2004

(54) Treatment of hepatitis C virus infection with sesquiterpene lactones

(76) Inventors: Hsing-Pang Hsieh, Taipei (TW); Tsu-An Hsu, Taipei (TW); Der-Ren Hwang, Keelung (TW)

Correspondence Address: FISH & RICHARDSON PC, 225 FRANKLIN ST, BOSTON, MA, 02110, US

(21) Appl. No.: 10/817,490

(22) Filed: Apr. 2, 2004

Related U.S. Application Data
(60) Provisional application No. 60/459,769, filed on Apr. 2, 2003.

(51) Int. Cl. A61K 31/365; A61K 38/21

(52) U.S. Cl. 514/450; 424/85.7

(57) ABSTRACT

This invention relates to a method for treating HCV infection. The method includes administering to a subject in need thereof an effective amount of one or more of sesquiterpene lactones having a γ-lactone fused with a 10-membered ring, an 8-membered ring that is further fused with a 4-membered ring, a 7-membered ring that is further fused with a 5-membered ring, or a 6-membered ring that is further fused with a 6-membered ring.

### CROSS REFERENCE TO RELATED APPLICATION

Pursuant to 35 U.S.C. § 119(e), this application claims priority to U.S. Provisional Application Ser. No. 60/459,769, filed Apr. 2, 2003.

### BACKGROUND

Chronic and acute hepatitis C virus (HCV) infection has caused serious medical, social, and economical problems. Patients with chronic HCV infection may develop liver diseases, including cirrhosis and hepatocellular carcinoma. It is estimated that HCV infection has infected 170 million individuals worldwide, among whom 20 to 30% may develop cirrhosis and 1 to 3% may develop liver cancer.

HCV infection has been treated with a combination of interferon-a and ribavirin (1-b-D-ribofuranosyl-1,2,4-triazole-3-carboxamide). Although this combination therapy represents a major advance in treating HCV infection, it is only effective in a limited fraction of patients (30-50%) and almost 50% of the individuals who respond to the treatment suffer a relapse. The low efficacy of this treatment has driven the search for more effective drugs.

### SUMMARY

The present invention is based on the unexpected discovery that certain sesquiterpene lactones are effective in treating HCV infection.

In one aspect, this invention features treating HCV infection by administering to a subject in need of the treatment an effective amount of a sesquiterpene lactone having a y-lactone fused with a 10-membered ring, fused with an 8-membered ring that is further fused with a 4-membered ring, fused with a 7-membered ring that is further fused with a 5-membered ring, or fused with a 6-membered ring that is further fused with a 6-membered ring. For example, one can administer to a subject having a HCV infection a sesquiterpene lactone, in which the y-lactone is substituted with a methylene group or an alkyl group.

“Treatment” refers to administering one or more sesquiterpene lactones to a subject, who has a HCV infection, a symptom of such an infection, or a predisposition toward such an infection, with the purpose to confer a therapeutic effect, e.g., to cure, relieve, alter, affect, ameliorate, or prevent the HCV infection, the symptom of it, or the predisposition toward it. Such a subject can be identified by a health care professional based on results from any suitable diagnostic method. “An effective amount” refers to the amount of one or more active sesquiterpene lactones that is required to confer a therapeutic effect on a treated subject. “Sesquiterpene lactones” include those present in natural resources, e.g. in a plant of the Compositae family, as well as those modified from naturally-occurring sesquiterpene lactones.

In particular, the invention features a method of treating a subject infected with HCV by administering to the subject an effective amount of a sesquiterpene lactone of the formula:

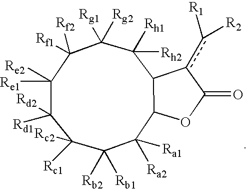

In this formula, each of R₁ and R₂, independently, is H, alkyl, cycloalkyl, heterocycloalkyl, aryl, heteroaryl, aralkyl, an amino acid moiety, a polypeptide moiety, F, Cl, Br, I, OR, SR, NRR′, C(O)R, COOR, or O(C)OR. [symbol] is a single bond or a double bond. Each of Rₐ₁ and Rₐ₂, independently, is H, alkyl, cycloalkyl, heterocycloalkyl, F, Cl, Br, I, OR, SR, C(O)R, COOR, or O(C)OR; or Rₐ₁ and Rₐ₂, taken together, is a methylene group, a carbonyl group, or an epoxy group; or one of Rₐ₁ and Rₐ₂, together with one of Rb, and R_{b2}, is a double bond, —CH₂—, or —O—; or one of Rₐ₁ and Rₐ₂, together with one of R_{d1} and R_{d2}, is a single bond or —O—; or one of Rₐ₁ and Rₐ₂, together with one of Rₑ₁ and Rₑ₂, is a single bond, —O—CR₂—O—, or —O—; or one of Rₐ₁ and Rₐ₂, together with one of R_{f1} and R_{f2}, is a single bond or —O—. Each of R_{b1} and R_{b2}, independently, is H, alkyl, cycloalkyl, heterocycloalkyl, F, Cl, Br, I, OR, SR, C(O)R, COOR, or O(C)OR; or R_{b1} and R_{b2}, taken together, is a methylene group, a carbonyl group, or an epoxy group; or one of R_{b1} and R_{b2}, together with one of R_{c1} and R_{c2}, is a double bond, —CH₂—, or —O—; or one of R_{b1} and R_{b2}, together with one of Rₑ₁ and Rₑ₂, is a single bond, —CRR′-CH₂—, or —O—; or one or R_{b1} and R_{b2}, together with one of R_{f1} and R_{f2}, is a single bond or —O—; or one or R_{b1} and R_{b2}, together with one of R_{g1} and R_{g2}, is a single bond or —O—. Each of R_{c1} and R_{c2}, independently, is H, alkyl, cycloalkyl, heterocycloalkyl, F, Cl, Br, I, OR, SR, C(O)R, COOR, or O(C)OR; or R_{c1} and R_{c2}, taken together, is a methylene group, a carbonyl group, or an epoxy group; or one of R_{c1} and R_{c2}, together with one of R_{d1} and R_{d2}, is a double bond, —CH₂—, or —O—; or one of R_{c1} and R_{c2}, together with one of R_{f1} and R_{f2}, is a single bond or —O—; or one of R_{c1} and R_{c2}, together with one of R_{g1} and R_{g2}, is a single bond or —O—; or one or R_{c1} and R_{c2}, together with one of Rₕ₁ and Rₕ₂, is a single bond or —O—. Each of R_{d1} and R_{d2}, independently, is H, alkyl, cycloalkyl, heterocycloalkyl, F, Cl, Br, I, OR, SR, C(O)R, COOR, or O(C)OR; or R_{d1} and R_{d2}, taken together, is a methylene group, a carbonyl group, or an epoxy group; or one of R_{d1} and R_{d2}, together with one of Rₑ₁ and Rₑ₂, is a double bond, —CH₂—, or —O—; or one of R_{d1} and R_{d2}, together with one of R_{f1} and R_{f2}, is —COO—; or one of R_{d1} and R_{d2}, together with one of R_{g1} and R_{g2}, is a single bond, —CRR′-CH₂—, or —O—; or one of R_{d1} and R_{d2}, together with one of Rₕ₁ and Rₕ₂, is a single bond or —O—. Each of Rₑ₁ and Rₑ₂, independently, is H, alkyl, cycloalkyl, heterocycloalkyl, F, Cl, Br, I, OR, SR, C(O)R, COOR, or O(C)OR; or Rₑ₁ and Rₑ₂, taken together, is a methylene group, a carbonyl group, or an epoxy group; or one of Rₑ₁ and Rₑ₂, together with one of R_{f1} and R_{f2}, is a double bond, —CH₂—, or —O—; or one of R_{c1} and R_{c2}, together with one of Rₕ₁ and Rₕ₂, is a single bond or —O—. Each of R_{f1} and R_{f2}, independently, is H, alkyl, cycloalkyl, heterocycloalkyl, F, Cl, Br, I, OR, SR, C(O)R, COOR, or O(C)OR; or R_{f1} and R_{f2}, taken together, is a methylene group, a carbonyl group, or an epoxy group; or one of R_{f1} and R_{f2}, together with one of R_{g1} and R_{g2}, is a double bond, —CH₂—, or —O—. Each of R_{g1} and R_{g2}, independently, is H, alkyl, cycloalkyl, heterocycloalkyl, F, Cl, Br, I, OR, SR, C(O)R, COOR, or O(C)OR; or R_{g1} and R_{g2}, taken together, is a methylene group, a carbonyl group, or an epoxy group; or one of R_{g1} and R_{g2}, together with one of Rₕ₁ and Rₕ₂, is a double bond, —CH₂—, or —O—. Each of Rₕ₁ and Rₕ₂, independently, is H, alkyl, cycloalkyl, heterocycloalkyl, F, Cl, Br, I, OR, SR, C(O)R, COOR, or O(C)OR; or Rₕ₁ and Rₕ₂, taken together, is a methylene group, a carbonyl group, or an epoxy group. Each of R and R′, independently, is H, hydroxy, aryl, alkyl, cycloalkyl, heterocycloalkyl; or R and R′, taken together, is a cycloalkyl or heterocycloalkyl.

For example, one can administer to a subject infected with HCV a sesquiterpene lactone of the above formula, in which each of R₁ and R₂ is H and [symbol] is a double bond. As another example, the subject is administered with a sesquiterpene lactone of the above formula, in which one of Rₐ₁ and Rₐ₂, together with one of Rₑ₁ and Rₑ₂, is a single bond; or one of Rₐ₁ and Rₐ₂, together with one of R_{f1} and R_{f2}, is a single bond; one or R_{b1} and R_{b2}, together with one of R_{g1} and R_{g2}, is a single bond; or one of R_{c1} and R_{c2}, together with one of R_{g1} and R_{g2}, is a single bond.

A subject in need of treatment of HCV infection can also be concurrently administered with a sesquiterpene lactone of the above formula and one or more other therapeutic agents. Examples of such therapeutic agents include IFNα, Intron A, PEG-INTRON, Roferon A, Pegasys, Infergen A, Wellferon, Omniferon, Interferon Omega, Albuferon-α, Rebif, Rebetron, Symmetrel, an NS2—NS3 autoprotease inhibitor, an NS3 protease inhibitor (e.g., BILN-2061 and VX-950/LY-570310), an NS3 helicase, an NS4 cofactor inhibitor, an NS5B polymerase inhibitor (JTK-003), an IRES inhibitor (e.g., ISIS 14803 and Heptazyme), an inosine monophosphate dehydrogenase inhibitor (e.g., VX-497, Levovirin, and Viramidine), an E2 inhibitor (e.g., XTL-002), an antifibrotic (e.g., Actimmune (IFN-©) and IP-501), a caspase inhibitor (e.g., IDN-6556), a β-tubulin inhibitor (e.g., T67), an anti-HCV IgG (e.g., Civacir), an immunosuppressant (e.g., CellCept), and an immune modulator (e.g., Ceplene and Zadazin). See e.g., Tan et al., Nature Review in Drug Discovery, (2002) 1:867. The term “concurrently administered” refers to administering a sesquiterpene lactone and one or more other therapeutic agents at the same time or at different times during a treatment period.

The term “alkyl” refers to a linear or branched, saturated or unsaturated, non-aromatic C₁-C₁₀ hydrocarbon moiety, e.g., CH₃ or CH═CHCH₃. The term “cycloalkyl” refers to a saturated or unsaturated C₃-C₂₀ cyclic hydrocarbon moiety. The term “heterocycloalkyl” refers to a saturated or unsaturated C₃-C₂₀ cyclic moiety having at least one ring heteroatom, such as O, N, and S. The term “aralkyl” refers to an alkyl group substituted with aryl or heteroaryl, e.g., benzyl or pyridinylmethyl. The term “aryl” refers to a hydrocarbon moiety having at least one aromatic ring. Examples of an aryl moiety include phenyl, pheyylene, naphthyl, naphthylene, pyrenyl, anthryl, and phenanthryl. The term “heteroaryl” refers to a moiety having at least one aromatic ring which contains at least one heteroatom, such as O, N, and S. Examples of a heteroaryl moiety include furyl, furylene, fluorenyl, pyrrolyl, thienyl, oxazolyl, imidazolyl, thiazolyl, pyridyl, pyrimidinyl, quinazolinyl, and indolyl.

Alkyl, cycloalkyl, heterocycloalkyl, aralkyl, aryl, and heteroaryl mentioned herein include both substituted and unsubstituted moieties. Examples of substituents for cycloalkyl, heterocycloalkyl, aralkyl, aryl, and heteroaryl include alkyl, cycloalkyl, heterocycloalkyl, alkoxy, cycloalkoxy, heterocycloalkoxy, aryl, aryloxy, heteroaryl, heteroaryloxy, alkylsulfonyl, arylsulfonyl, heteroarylsulfonyl, amino, alkylamino, dialkylamino, arylamino, diarylamino, alkylimino, arylimino, amido, carbamoyl, thioamido, thiocarbamoyl, hydroxyl, halogen, mercapto, alkylmercapto, arylmercapto, cyano, nitro, acyl, acyloxy, carboxyl, and carboxylic esters. Examples of substituents for alkyl include all of the above substitutents except alkyl. Cycloalkyl, heterocycloalkyl, aryl, and heteroaryl also include fused groups.

Shown below are exemplary sesquiterpene lactones that can be used to practice the method of the invention:

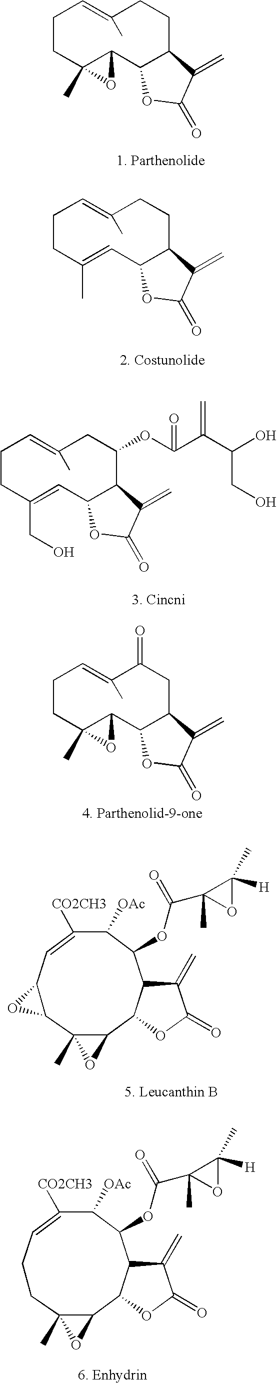

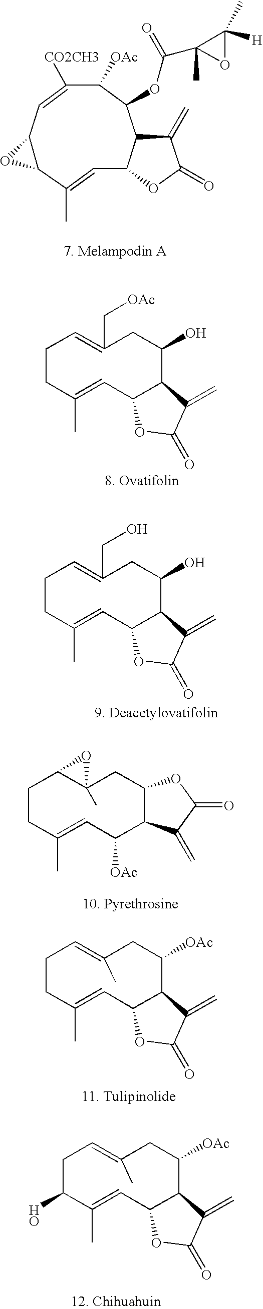

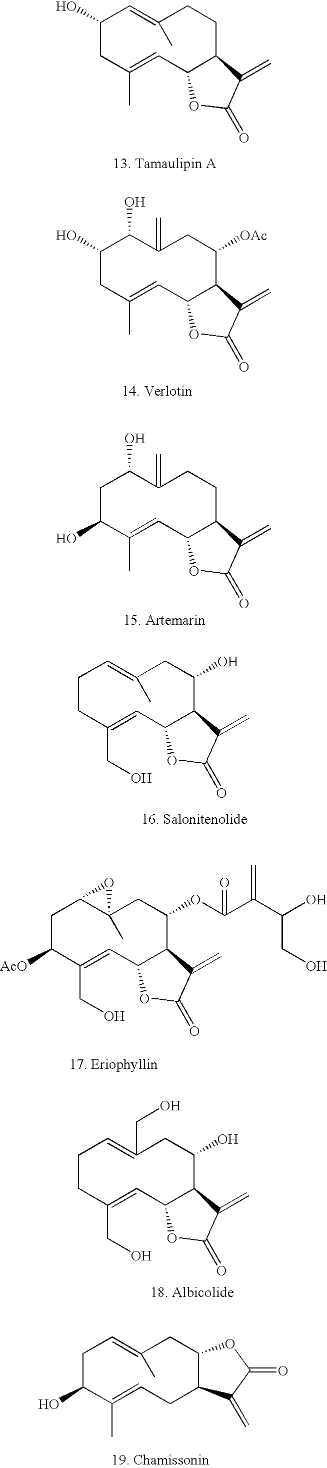

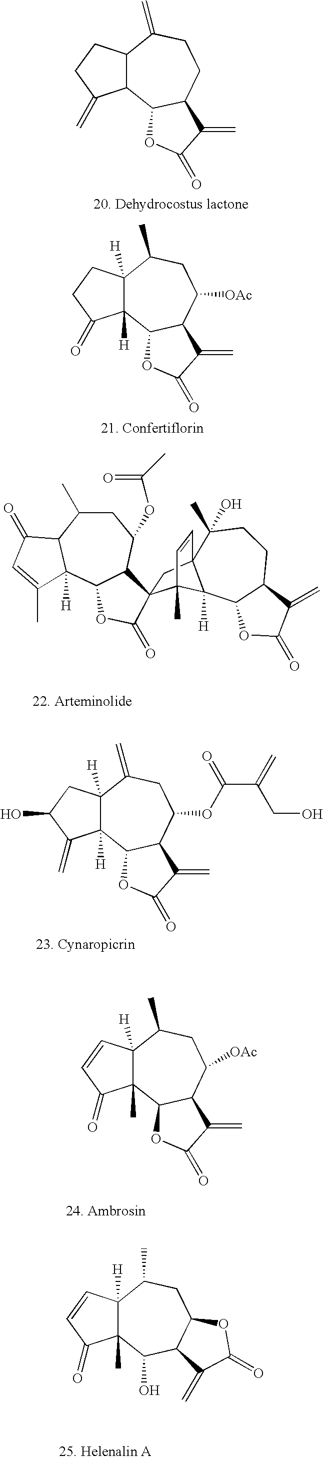

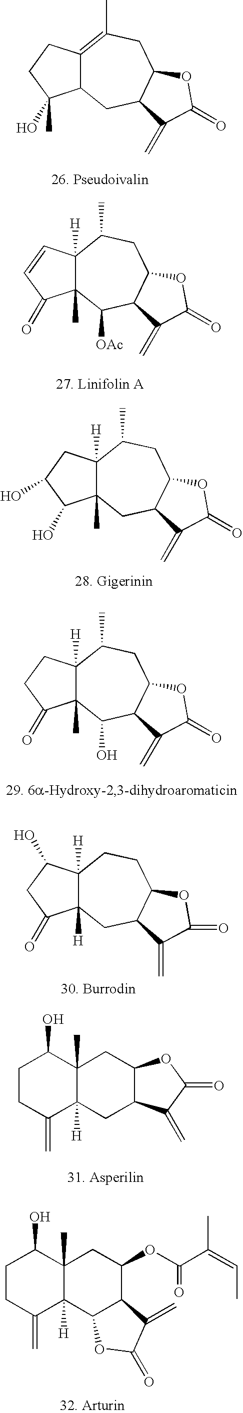

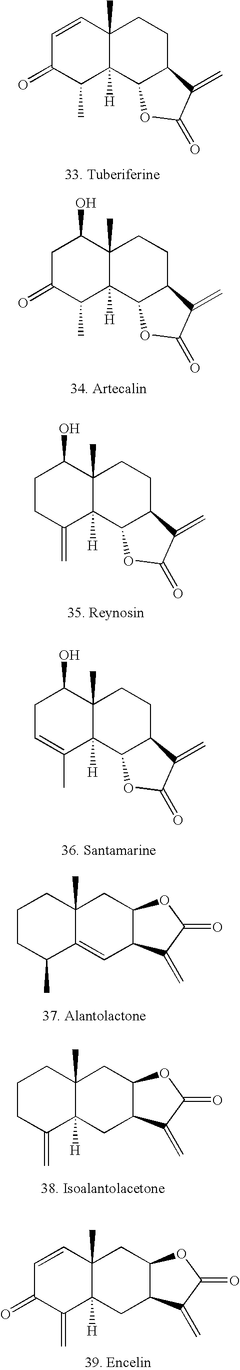

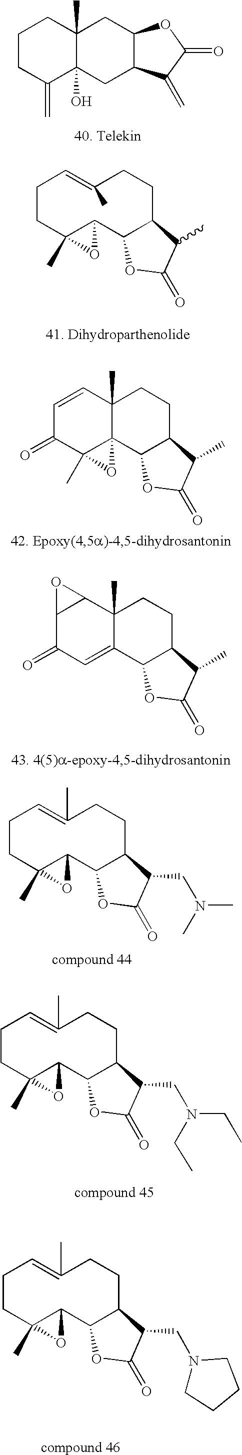

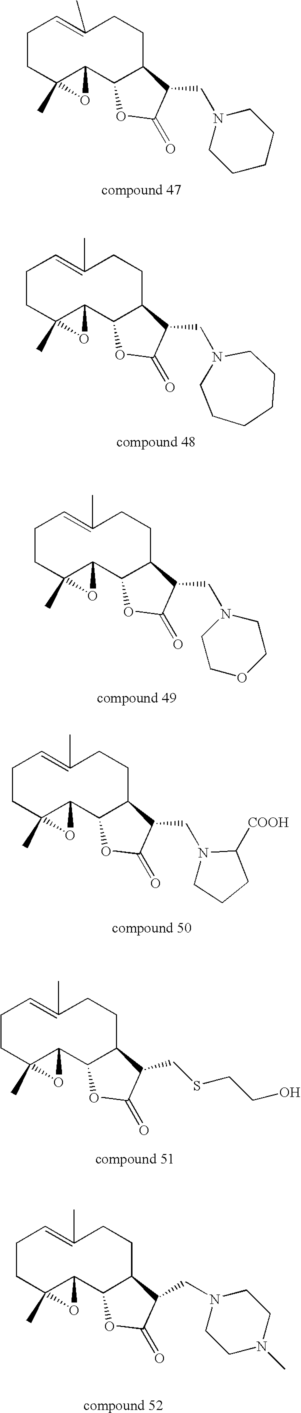

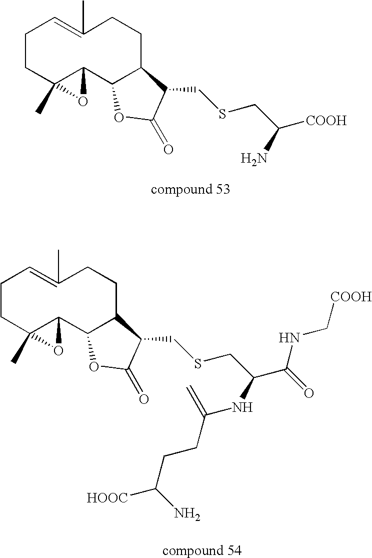

The sesquiterpene lactones described above include the compounds themselves, as well as their salts and their prodrugs, if applicable. A salt, for example, can be formed between an anion and a positively charged group (e.g., amino) on a sesquiterpene lactone. Suitable anions include chloride, bromide, iodide, sulfate, bisulfate, sulfamate, nitrate, phosphate, citrate, methanesulfonate, trifluoroacetate, maleate, succinate, fumarate, tartrate, salicylate, lactate, naphthalenesulfonate, and acetate. Likewise, a salt can also be formed between a cation and a negatively charged group (e.g., carboxylate) on a sesquiterpene lactone. Suitable cations include sodium ion, potassium ion, magnesium ion, calcium ion, and an ammonium cation such as tetramethylammonium ion. The sesquiterpene lactones also include those salts containing quaternary nitrogen atoms. Examples of prodrugs include esters and other pharmaceutically acceptable derivatives, which, upon administration to a subject, are capable of providing active sesquiterpene lactones.

Also within the scope of this invention is a composition containing one or more of the sesquiterpene lactones described above for use in treating HCV infection, and the use of such a composition for the manufacture of a medicament for the just-mentioned treatment.

The details of the embodiments of the invention are set forth in the description below. Other features, objects, and advantages of the invention will be apparent from the following description and from the claims.

### DETAILED DESCRIPTION

This invention relates to use of one or more sesquiterpene lactones described in the summary section above for treating HCV infection. The sesquiterpene lactones can be found naturally or modified from naturally-occurring sesquiterpene lactones. Naturally-occurring sesquiterpene lactones can be obtained by extracting them from plants (e.g., of the Compositae family). For example, extraction is carried out by squeezing juice out of leaves of a Compositae plant. Alternatively, it can be carried out by immersing pulverized leaves in an organic solvent, such as ethanol, dichloromethane, or hexane. In the latter case, a crude extract can be produced either by a batch method or by a continuous method. The crude extract thus obtained is filtered or centrifuged to remove any insoluble materials. A sesquiterpene lactone is then isolated from the crude extract using liquid chromatography (e.g., high-pressure liquid chromatography) or other suitable methods.

Naturally-occurring sesquiterpene lactones can also be modified by synthetic methods well known in the chemical art. The scheme below depicts the syntheses of exemplary sesquiterpene lactones, i.e., compounds 41 and 44-54. Details of preparation of these compounds are provided in Examples 1-12, respectively.

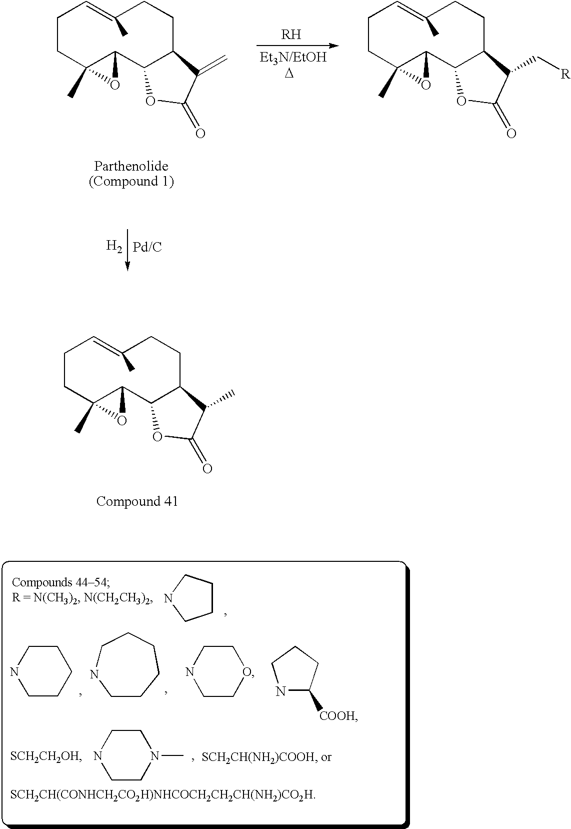

As an example, the methylene group on y-lactone ring of parthenolide (a naturally-occurring sesquiterpene lactone) can be reduced to form a methyl group using a suitable reducing agent. (Kwok et al., Chem Biol (2001) 8(8): 759-66.) As another example, parthenolide can be modified with a secondary amine (e.g., dimethylamine) or a sulfur-containing compound (such as mercaptoethanol, cystine, or a cystine-containing peptide). Methods for obtaining various sesquiterpene lactones can be found in references 1-180 listed below.

The sesquiterpene lactones mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition contains an effective amount of at least one sesquiterpene lactone described above and a pharmaceutical acceptable carrier. This invention also covers a method of administering an effective amount of one or more sesquiterpene lactones to treat HCV infection. Effective doses will vary, as recognized by those skilled in the art, depending on the route of administration, excipient usage, and the possibility of co-usage with other therapeutic treatment.

To practice the method of the present invention, a composition having one or more sesquiterpene lactones can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions, and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more active sesquiterpene lactones can also be administered in the form of suppositories for rectal administration.

A pharmaceutically acceptable carrier is routinely used with one or more active sesquiterpene lactones. The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active sesquiterpene lactone. Examples of other carriers include colloidal silicon oxide, magnesium stearate, cellulose, sodium lauryl sulfate, and D&C Yellow # 10.

Sesquiterpene lactones can be preliminarily screened for their efficacy in treating HCV infection by the following in vitro assays (See Examples 13, 14, and 15 below) and further screened by in vivo assays. (Yeh et al., J. Virol., (2001) 75:11017-11024.) Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 41: 4,8,12-Trimethyl-3,14-dioxa-tricyclo-[9.3.0.0^{2,4}]tetradec-7-en-13-one

The title compound was obtained in 95% yield by reducing parthenolide (compound 1) in hydrogen gas in the presence of catalytic palladium on charcoal.

¹H NMR (300 MHz, CDCl₃) 6 (ppm): 1.16-2.46 (m, 19H), 2.71 (d, J=9.3 Hz, 1H), 3.81 (t, J=9 Hz, 1H), 5.17 (dd, J=2.1, 12 Hz, 1H).

### EXAMPLE 2

### Preparation of Compound 44: 12-Dimethylaminomethyl-4,8-dimethyl-3,14-dioxa-tricyclo[9.3.0.02,4]tetradec-7-en-13-one

Parthenolide (50 mg, 0.20 mmol) and dimethylamines (0.22 mmol) were dissolved in EtOH (6 mL) and the solution was stirred at room temperature overnight. The reaction mixture was quenched by adding water (3 mL) and extracted with ethyl acetate (3×20 mL). The organic layers were combined, dried, and concentrated in vacuo to give a residue, which was purified by silica gel column chromatography (AcOEt: n-hexane=1:1) to afford the title compound.

¹H NMR (300 MHz, CDCl₃) 6 (ppm): 1.17-2.46 (m, 23H), 2.68 (ddd, J=4.6, 13.5, 36.3 Hz, 1H), 2.74 (d, J=9.3 Hz, 1H), 3.82 (t, J=9 Hz, 1H), 5.19 (dd, J=2.4, 12 Hz, 1H).

HRMS (EI) for C₁₇H₂₇NO₃ (M+): calculated: 293.1991; found: 293.1992.

### EXAMPLE 3

### Preparation of Compound 45: 12-Diethylaminomethyl-4,8-dimethyl-3,14-dioxa-tricyclo[9.3.0.02,4]tetradec-7-en-13-one

The title compound was prepared following the procedure described in Example 2 in the presence of diethylamine

¹H NMR (300 MHz, CDCl₃) 6 (ppm): 1.01 (t, J=7.2 Hz, 6H), 1.17-2.59 (m, 20H), 2.72 (d, J=9 Hz, 1H), 2.80 (ddd, J=4.9, 14.1, 29.8 Hz, 2H), 3.81 (t, J=9 Hz, 1H), 5.15 (dd, J=2.4, 12 Hz, 1H).

HRMS (EI) for C₁₉H₃₁NO₃ (M+): calculated: 321.2304; found: 321.2309.

### EXAMPLE 4

### Preparation of Compound 46: 4,8-Dimethyl-12-pyrrolidin-1-ylmethyl-3,14-dioxa-tricyclo[9.3.0.02,4]tetradec-7-en-13-one

The title compound was prepared following the procedure described in Example 2 in the presence of pyrrolidine.

¹H NMR (300 MHz, CDCl₃) 6 (ppm): 1.17-1.33 (m, 24H), 2.74 (d, J=9 Hz, 1H), 2.89 (qd, J=4.6, 12.6 Hz, 2H), 3.82 (t, J=8.85 Hz, 1H), 5.19 (dd, J=2.4, 12 Hz, 1H).

HRMS (E1) for C₁₉H₂₉NO₃ (M): calculated: 319.2147; found: 319.2142.

### EXAMPLE 5

### Preparation of Compound 47: 4,8-Dimethyl-12-piperidin-1-ylmethyl-3,14-dioxa-tricyclo[9.3.0.02,4]tetradec-7-en-13-one

The title compound was prepared following the procedure described in Example 2 in the presence of piperidine.

¹H NMR (300 MHz, CDCl₃) 6 (ppm): 1.18-2.49 (m, 26H), 2.70 (ddd, J=5.3, 13.4, 43.8 Hz, 2H), 2.71 (d, J=8.7 Hz, 1H), 3.82 (t, J=9 Hz, 1H), 5.18 (dd, J=2.2, 11.8 Hz, 1H).

HRMS (EI) for C₂₀H₃₁NO₃ (M+): calculated: 333.2304; found: 333.2305.

### EXAMPLE 6

### Preparation of Compound 48: 12-Azepan-1-ylmethyl-4,8-dimethyl-3,14-dioxa-tricyclo[9.3.0.02,4]tetradec-7-en-13-one

The title compound was prepared following the procedure described in Example 2 in the presence of 1-aminohomopiperidine.

¹H NMR (300 MHz, CDCl₃) 6 (ppm): 1.18-2.74 (m, 29H), 2.92 (t, J=4.0 Hz, 2H), 3.82 (t, J=8.8 Hz, 1H), 5.19 (dd, J=2.1, 12 Hz, 1H).

HRMS (E1) for C₂₁H₃₃NO₃ (M+): calculated 347.2460; found: 347.2507.

### EXAMPLE 7

### Preparation of Compound 49: 4,8-Dimethyl-12-morpholin-4-ylmethyl-3,14-dioxa-tricyclo[9.3.0.02,4]tetradec-7-en-13-one

The title compound was prepared following the procedure described in Example 2 in the presence of morpholine and purified using column chromatography (AcOEt: n-hexane=2:1).

¹H NMR (300 MHz, CDCl₃) 6 (ppm): 1.18-2.56 (m, 20H), 2.72 (d, J=9 Hz, 1H), 2.75 (ddd, J=5.4, 13, 26.1 Hz, 2H), 3.60 (t, J=4.5 Hz, 4H), 3.83 (t, J=9 Hz, 1H), 5.19 (dd, J=2.4, 12 Hz, 1H).

HRMS (EI) for C₁₉H₂₉NO₄ (M+): calculated: 335.2097; found: 335.2098.

### EXAMPLE 8

### Preparation of Compound 50: 1-(4,8-Dimethyl-13-oxo-3,14-dioxa-tricyclo[9.3.0.02,4]tetradec-7-en-12-ylmethyl)-pyrrolidine-2-carboxylic acid

The title compound was prepared following the procedure described in Example 2 in the presence of L-proline and purified by using column chromatography (CH₂Cl₂: MeOH: H₂O=15:4:1).

¹H NMR (300 MHz, CDCl₃) 8 (ppm): 1.21-2.49 (m, 19H), 2.91 (d, J=9.3 Hz, 1H), 3.07-3.71 (m, 4H), 3.84 (ddd, J=3, 7.5, 10.8 Hz, 1H), 4.00 (dd, J=4.8, 9.3 Hz, 1H), 4.15 (t, J=4.8 Hz, 1H), 5.26 (dd, J=2.1, 12 Hz, 1H).

HRMS (E) for C₂₀H₂₉NO₅ (M⁺-COOH): calculated: 318.2069; found: 318.2082.

### EXAMPLE 9

### Preparation of Compound 51: 12-(2-Hydroxy-ethylsulfanylmethyl)-4,8-dimethyl-3,14-dioxa-tricyclo[9.3.0.02,4]tetradec-7-en-13-one

The title compound was prepared following the procedure described in Example 2 in the presence of 2-mercaptoethanol and purified by using column chromatography (CH₂Cl₂: MeOH=15:1).

¹H NMR (300 MHz, CDCl₃) 8 (ppm): 1.20˜2.84 (m, 20H), 3.00 (ddd, J=4.5, 13.8, 30 Hz, 2H), 3.79 (t, J=5.4 Hz, 2H), 3.87 (t, J=9 Hz, 1H), 5.21 (dd, J=2.1, 12 Hz, 1H).

HRMS (EI) for C₂₀H₂₉NO₅ (C₁₇H₂₆O₄S): calculated: 326.1552; found: 326.1542.

### EXAMPLE 10

### Preparation of Compound 52: 4,8-Dimethyl-12-(4-methyl-piperazin-1-ylmethyl)-3,14-dioxa-tricyclo[9.3.0.02,4]tetradec-7-en-13-one

The title compound was prepared following the procedure described in Example 2 in the presence of N-methylpiperazine and purified by using column chromatography (CH₂Cl₂: MeOH=15:1).

¹H NMR (300 MHz, CDCl₃) (ppm): 1.18-2.54 (m, 27H), 2.71 (d, J=8.7 Hz, 1H), 2.76 (ddd, J=4.9, 13.6, 29.2 Hz, 2H), 3.82 (t, J=9 Hz, 1H), 5.19 (dd, J=2.1, 12 Hz, 1H).

HRMS (EI) for C₂₀H₃₂N₂O₃ (M+): calculated: 348.2413; found: 348.2420.

### EXAMPLE 11

### Preparation of Compound 53: 2-Amino-3-(4,8-dimethyl-13-oxo-3,14-dioxa-tricyclo[9.3.0.02,4]tetradec-7-en-12-ylmethylsulfanyl)-propionic acid

The title compound was prepared following the procedure described in Example 2 in the presence of cysteine.

¹H NMR (300 MHz, CDCl₃) (ppm): 1.08-3.24 (m, 21H), 3.66 (dd, J=3.7, 7.9 Hz, 1H), 3.94 (t, J=9.1 Hz, 1H), 5.16 (dd, J=2.1, 12 Hz, 1H).

HRMS (E1) for C₁₈H₂₇NO₅S (M+): calculated: 369.1610; found: 369.1654.

### EXAMPLE 12

### Preparation of Compound 54: 2-Amino-4-[1-(carboxymethyl-carbamoyl)-2-(4,8-dimethyl-13-oxo-3,14-dioxa-tricyclo[9.3.0.02,4]tetradec-7-en-12-ylmethylsulfanyl)-ethylcarbamoyl]-butyric acid

The title compound was prepared following the procedure described in Example 2 in the presence of a glycine-cysteine-glutamine peptide.

¹H NMR (300 MHz, CDCl₃) (ppm): 1.21-3.18 (m, 32H), 3.91 (s, 2H), 4.23 (t, J=9 Hz, 1H), 4.62 (dd, J=4.9, 8.5 Hz, 1H), 5.28 (dd, J=2.1, 12 Hz, 1H).

HRMS (EI) for C₂₅H₃₇N₃O₉S (M⁺): calculated: 555.2251; found: 555.2243.

### EXAMPLE 13

### Inhibition of Cell Growth

Compounds 1, 2, 20, 25, 37, 38, 41, 44-51, 53, and two control compounds (i.e. α-methylene-γ-lactone and isotonic anhydrate) were tested for their cytotoxicity using the following assay.

Huh-7 cells containing HCV sub-genomic replicons (Ava.5) were provided by Apath, LLC. (St. Louis, Mo.). See, e.g., Blight et al. (2000) Science 290: 1972-4. Cells were maintained in Dulbecco's modified Eagle's medium (DMEM) supplemented with 10% fetal calf serum (FCS) in a humidified atmosphere containing 5% of CO₂. Cell viability was determined using 3-(4,5-dimethylthiazol-2-yl)-5-(3-carboxymethoxyphenyl)-2-(4-sulfophenyl)-2H-tetrazolium, inner salt (MTS), as described in Malich et al. (1997) Toxicology 124: 179-92. MTS, in the presence of phenazine methosulfate (PMS), produced a water-soluble formazan product that had an absorbance maximum at 490-500 nm in phosphate-buffered saline (PBS). See, e.g., Cory et al. (1991) Cancer Commun., 3:207-12.

MTS and PMS, both in powder form, were purchased from Sigma or Promega. MTS was dissolved in PBS (2 mg/mL) and PMS (0.38 mg/mL) was dissolved in ddH₂O as stock solutions. Cells cultured in a 96-well microtiter plate were treated with the eight sesquiterpene lactones and the two control compounds at various concentrations. Prior to the assay, a 2 mL MTS/PMS solution containing MTS and PMS (20:1) was added to the cells with 8 mL of phenol red-free DMEM. The cell culture medium was then aspirated off, washed with PBS, and 100 μL of the MTS/PMS mixture was added into each well. After incubation at room temperature for 2 hr, optical densities of each sample were measured at OD₄₉₀ with a 96-well microtiter plate reader. Data were obtained with six repeats for each treatment.

Each of the test compounds showed an IC₅₀ value (i.e., the concentration at which cell growth was inhibited by 50%) greater than 10 μM except for compounds 25 and 50, which showed IC₅₀ values of 1.6 μM and 8.1 μM, respectively.

### EXAMPLE 14

### Inhibition of HCV RNA Replication

Compounds 1, 2, 20, 25, 37, 41, 44-50, 53, and the two control compounds were tested at non-cytotoxic doses for their efficacy in inhibiting HCV infection.

Huh-7 cells containing HCV sub-genomic replicons (Ava.5) were maintained in DMEM supplemented with 10% heat-inactivated FCS in a humidified atmosphere containing 5% of CO₂. The medium also contained 1 mg/mL G418 (an aminoglycosidic antibiotic agent obtained from Promega, WI) as a selective pressure for the maintenance of HCV replicon's RNA replication. See, e.g., Lohmann et al. (2001) J. Virol. 75: 1437-49. Cells were treated with sesquiterpene lactones and control compounds at various concentrations. Then, cells were harvested after 24 hr of the treatment. Subsequently, total cellular RNA's were extracted and quantified as follows.

Total RNA of cells was extracted using RNeasy Mini Kit (QIAGEN). The concentration of total RNA was measured by absorption at 260 nm. Total RNA (1 μg) was then mixed with 50 picomoles of HCV specific reverse primer: A9412, 5′-GATGGC CTATTG GCCTGG AGGGG-3′ (Lohmann et al. (2001) J. Virol. 75: 1437-49). RNA was added with RNase-free ddH₂O to a final volume of 10.5 μL and denatured for 10 min at 65° C. Reverse transcription (RT) reactions were carried out at 42° C. for 1 hr using Expand-RT (Roche Biochemicals, Mannheim, Germany) in a total volume of 20 μL containing a previously denatured primer-RNA mixture, 1 mM each of deoxynucleotide triphosphates (dNTPs), 50 units reverse transcriptase, and 20 units RNase inhibitor (Promega, Madison, Wis.).

Quantitative PCR (Q-PCR) was carried out by LightCycler-DNA Master using SYBR Green 1 for real-time detection of double-stranded DNA (dsDNA) (Roche Diagnostics GmbH, Mannheim, Germany). The intensity of fluorescence was recorded versus PCR cycle numbers and the relative amounts of HCV RNA were calculated corresponding to the standard curve. The standard curve was derived from five serial dilutions of reference DNA, the corresponding cDNA of HCV RNA, under criteria recommended by the supplier.

The relative copy numbers HCV RNA and a house-keeping gene, glyceraldehyde-3-phosphate dehydrogenase (GADPH), were determined by Q-PCR utilizing the following primer sets:

HCV primers (positive strand):

| | | |
|---|---|---|
| Forward: | 5′-ACCAGAATACGACTTGGAGTTGAT | (SEQ ID NO:1) |
| | AA-3′ | |
| Reverse: | 5′-CACCCTTTTGCCAGATGCAT-3′ | (SEQ ID NO:2) |

HCV primers (negative strand):

| | | |
|---|---|---|
| Forward: | 5′-CTCGACGTTGTCACTGA-3′ | (SEQ ID NO:3) |
| Reverse: | 5′-CCATCCGAGTACGTGC-3′ | (SEQ ID NO:4) |

GADPH primers

| | | |
|---|---|---|
| Forward primer: | 5′-GAAGGTGAAGGTCGGAG | (SEQ ID NO:5) |
| | TC-3′ | |
| Reverse primer: | 5′-GAAGATGGTGATGGGATT | (SEQ ID NO:6) |
| | TC-3′ | |

PCR was performed as followed: (i) denaturing conditions: 95° C., 60 sec; (ii) amplification conditions: ramp to 95° C., 0 sec; 64° C., 10 sec; 72° C., 10 sec. The specificity of an amplification reaction was determined by performing a melting curve analysis with a temperature range from 65° C. to 95° C.

HCV RNA copy numbers were divided by their corresponding GADPH copy numbers in the same sample for normalization. Effect of sesquiterpene lactones on HCV RNA replication was represented by “relative copy number,” which was obtained by comparing HCV RNA copy numbers with sesquiterpene lactones treatment to HCV RNA copy numbers without sesquiterpene lactones treatment. The latter was designated as 100%. All measurements were performed in triplicates to reach statistical significance. Sesquiterpene lactones inhibited replication of HCV RNA positive strand in a dose-dependent manner.

Unexpectedly, each of compounds 1, 2, 20, 25, 37, 44-48, and 50 was found to have an EC₅₀ value (i.e., the effective concentration at which HCV RNA replication was inhibited by 50%) no greater than 3 μM. Each of compounds 49 and 53 was found to have an EC₅₀ value no greater than 6 μM.

### EXAMPLE 15

Compound 1, interferon-α (IFNα), and a combination of compound 1 and IFNα were tested for their efficacy in inhibiting HCV infection using the HCV RNA replication assay described above.

It was found that compound 1 inhibited HCV RNA replication in a dose dependent manner. At concentration 0.625 μM, 1.25 μM, 2.5 μM, and 5 μM, compound 1 inhibited 37%, 47%, 70%, and 83% of HCV RNA replication, respectively.

It was also found that the combination of compound 1 and IFNα was more effective in inhibiting HCV RNA replication than IFNα alone. More specifically, 50 IU/1 mL and 100 IU/mL of IFNα alone respectively inhibited 25% and 40% of HCV RNA replication. In contrast, 50% and 70% of HCV RNA replication were inhibited by 50 IU/mL IFNα combined with 0.1 μM compound 1 and 100 IU/mL of IFNα combined with 0.1 μM compound 1, respectively.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

### REFERENCES

1. Chhabra, B. R.; Jain, Meenu; Indian Journal of Chemistry, Section B: Organic Chemistry Including Medicinal Chemistry (2002), 41B(8), 1744-1746.

2. Yang, Chao; Shi, Yan-Ping; Jia, Zhong-Jian; Planta Medica (2002), 68(7), 626-630.

3. Vargas, David; Fischer, Nikolaus H.; Younathan, Ezzat S.; Revista de la Sociedad Quimica de Mexico (2001), 45(4), 159-162.

4. Babish, John G.; Howell, Terrence; Pacioretty, Linda; U.S. Pat. Appl. Publ. 2002077299, 18 pp.

5. Lang, Gunter; Passreiter, Claus M.; Wright, Colin W.; Filipowicz, Natalia H.; Addae-Kyereme, Jonathan; Medinilla, Beatriz E.; Castillo, Juan-Jose; Zeitschrift fuer Naturforschung, C: Journal of Biosciences (2002), 57(3/4), 282-286.

6. Kasenov, B. K.; Adekenov, S. M.; Mustafin, E. S.; Tukhmetova, Zh. K.; Kulyyasov, A. T.; Zhurnal Fizicheskoi Khimii (2002), 76(2), 206-209.

7. Mata, Rachel; Rivero-Cruz, Isabel; Rivero-Cruz, Blanca; Bye, Robert; Timmermann, Barbara N.; Journal of Natural Products (2002), 65(7), 1030-1032.

8. De Kraker, Jan-Willem; Franssen, Maurice C. R.; Joerink, Maaike; De Groot, Aede; Bouwmeester, Harro J.; Plant Physiology (2002), 129(1), 257-268.

9. Yang, Chao; Shi, Yan Ping; Wang, Xing; Jia, Zhong Jian; Chinese Chemical Letters (2002), 13(3), 247-248.

10. Bishay, D. W.; Attia, A. A.; Fayed, M. A.; Bulletin of Pharmaceutical Sciences, Assiut University (2001), 24(2), 243-249.

11. Galindo, Juan C. G.; de Luque, Alejandro P.; Jorrin, Jesus; Macias, Francisco A.; Journal of Agricultural and Food Chemistry (2002), 50(7), 1911-1917.

12. Ha, Tae Joung; Yang, Min Suk; Pak, Yunbae; Lee, Jong Rok; Lee, Kyung Dong; Kim, Hwan Mook; Park, Ki Hun; Heterocycles (2002), 57(1), 151-155.

13. Todorova, Milka; Tsankova, Elena; Zeitschrift fuer Naturforschung, C: Journal of Biosciences (2001), 56(11/12), 957-960.

14. Lang, Gunter; Passreiter, Claus M.; Medinilla, Beatriz E.; Castillo, Juan-Jose; Pharmaceutical Biology (Lisse, Netherlands) (2001), 39(5), 332-335.

15. Ganzera, M.; Mair, M.; Stuppner, H.; Fischer, N. H.; Khan, I. A.; Chromatographia (2001), 54(9/10), 665-668.

16. Janackovic, Pedja; Tesevic, Vele; Marin, Petar D.; Milosavljevic, Slobodan M.; Petkovic, Branimir; Sokovic, Marina; Biochemical Systematics and Ecology (2002), 30(1), 69-71.

17. Fuchino, Hiroyuki; Koide, Tatsuo; Takahashi, Marii; Sekita, Setsuko; Satake, Motoyoshi; Planta Medica (2001), 67(7), 647-653.

18. Jean, Daniel; Cariel, Leon; PCT Int. Appl., WO 2001076551, 26 pp.

19. Michalska, K.; Kisiel, W.; Polish Journal of Chemistry (2001), 75(10), 1587-1589.

20. Jean, Daniel; Cariel, Leon; PCT Int. Appl., WO 2001060326, 18 pp.

21. Heilmann, J.; Wasescha, M. R.; Schmidt, T. J.; Bioorganic & Medicinal Chemistry (2001), 9(8), 2189-2194.

22. Fortuna, A. M.; de Riscala, E. C.; Catalan, C. A. N.; Gedris, T. E.; Herz, W.; Biochemical Systematics and Ecology (2001), 29(9), 967-971.

23. Triana, J.; Eiroa, J. L.; Lopez, M.; Ortega, J. J.; Gonzalez, A. G.; Barrera, J. B.; Biochemical Systematics and Ecology (2001), 29(8), 869-871.

24. Adekenov, S. M.; Vasin, Yu. A.; Kalashnikova, T. S.; Samsonova, A. N.; Khimiya Prirodnykh Soedinenii (1992), (6), 637-640.

25. Liu, Changhong; Mishra, A. K.; He, Bing; Tan, Renxiang; Chinese Science Bulletin (2001), 46(6), 498-501.

26. Ju Kim, E.; Jin, H. K.; Kim, Y. K.; Lee, H. Y.; Lee, S. Y.; Lee, K. R.; Zee, O. P.; Han, J. W.; Lee, H. W.; Biochemical Pharmacology (2001), 61(7), 903-910.

27. Bruno, Maurizio; Maggio, Antonella; Paternostro, Maria Pia; Rosselli, Sergio; Arnold, Nelly A.; Herz, Werner; Biochemical Systematics and Ecology (2001), 29(4), 433435.

28. Kisiel, W.; Zielinska, K.; Polish Journal of Chemistry (2001), 75(1), 141-145.

29. Wang, Yongbing; Xu, Hua; Zhang, Yufeng; Wang, Qiang; Yaowu Fenxi Zazhi (2000), 20(6), 366-368.

30. Gonzalez-Romero, Maria A.; Villaescusa-Castillo, Lucinda; Diaz-Lanza, Ana M.; Zeitschrift fuer Naturforschung, C: Journal of Biosciences (2000), 55(9/10), 697-700.

31. De la Fuente, J. R.; Uriburu, M. L.; Burton, G.; Sosa, V. E.; Phytochemistry (2000), 55(7), 769-772.

32. Skaltsa, H.; Lazari, D.; Panagouleas, C.; Georgiadou, E.; Garcia, B.; Sokovic, M.; Phytochemistry (2000), 55(8), 903-908.

33. Milovanovic, Mirjana; Picuric-Jovanovic, Ksenija; Djermanovic, Miodrag; Djermanovic, Verica; Stefanovic, Milutin; Journal of the Serbian Chemical Society (2000), 65(11), 763-767.

34. Dirsch, V. M.; Stuppner, H.; Ellmerer-Muller, E. P.; Vollmar, A. M.; Bioorganic & Medicinal Chemistry (2000), 8(12), 2747-2753.

35. Orabi, Khaled Y.; El Sayed, Khalid A.; Al-Said, Mansour S.; El-Feraly, Farouk S.; Saudi Pharmaceutical Journal (2000), 8(2-3), 101-105.

36. Narkunan, Kesavaram; Shiu, Lin-Hun; Liu, Rai-Shung; Synlett (2000), (9), 1300-1302.

37. Jung, Chil Mann; Kwon, Hak Cheol; Choi, Sang Zin; Lee, Jae Hoon; Lee, Dong Jin; Ryu, Su No; Lee, Kang Ro; Saengyak Hakhoechi (2000), 31(2), 125-129.

38. Skaltsa, Helen; Lazari, Diamanto; Garcia, Begona; Pedro, Jose R.; Sokovic, Marina; Constantinidis, Theophanis; Zeitschrift fuer Naturforschung, C: Journal of Biosciences (2000), 55(7/8), 534-539.

39. Dendougui, H.; Benayache, S.; Benayache, F.; Connoly, J. D.; Fitoterapia (2000), 71(4), 373-378.

40. Todorova, M.; Ognyanov, I.; Dokladi na Bulgarskata Akademiya na Naukite (1999), 52(34), 41-44.

41. Lee, S.-H.; Kang, H.-M.; Song, H.-C.; Lee, H.; Lee, U. C.; Son, K.-H.; Kim, S.-H.; Kwon, B.-M.; Tetrahedron (2000), 56(27), 4711-4715.

42. Alarcon, R.; Vaccarini, C.; Sosa, V.; Molecules [Electronic Publication] (2000), 5(3), 433-434.

43. Cho, J. Y.; Baik, K. U.; Jung, J. H.; Park, M. H.; European Journal of Pharmacology (2000), 398(3), 399-407.

44. Recio, M. C.; Giner, R. M.; Uriburu, L.; Manez, S.; Cerda, M.; De la Fuente, J. R.; Rios, J. L.; Life Sciences (2000), 66(26), 2509-2518.

45. Jodynis-Liebert, Jadwiga; Murias, Marek; Bloszyk, Elzbieta; Planta Medica (2000), 66(3), 199-205.

46. Wedge, D. E.; Galindo, J. C. G.; Macias, F. A.; Phytochemistry (2000), 53(7), 747-757.

47. Barrero, Alejandro F.; Oltra, J. Enrique; Alvarez, Miriam; Raslan, Delio S.; Saude, Denia A.; Akssira, Mohammed; Fitoterapia (2000), 71(1), 60-64.

48. Mazor, Robert L.; Menendez, Ingrid Y.; Ryan, Marnie A.; Fiedler, Michael A.; Wong, Hector R.; Cytokine (2000), 12(3), 239-245.

49. Guillet, Gabriel; Harmatha, Juraj; Waddell, Thomas G.; Philogene, Bernard J. R.; Arnason, John T.; Photochemistry and Photobiology (2000), 71(2), 111-115.

50. Schmidt, Thomas J.; Willuhn, Gunter; Biochemical Systematics and Ecology (2000), 28(2), 133-142.

51. De Luque, Alejandro Perez; Galindo, Juan Carlos G.; Macias, Francisco A.; Jorrin, Jesus; Phytochemistry (2000), 53(1), 45-50.

52. Schmidt, Thomas J.; Lyss, Guido; Pahl, Heike L.; Merfort, Irmgard; Bioorganic & Medicinal Chemistry (1999), 7(12), 2849-2855.

53. Berdin, A. G.; Adekenov, S. M.; Raldugin, V. A.; Shakirov, M. M.; Druganov, A. G.; Kulyyasov, A. T.; Tolstikov, G. A.; Russian Chemical Bulletin (Translation of Izvestiya Akademii Nauk, Seriya Khimicheskaya) (1999), 48(10), 1987-1991.

54. Rungeler, Peter; Castro, Victor; Mora, Gerardo; Goren, Nezhun; Vichnewski, Walter; Pahl, Heike L.; Merfort, Irmgard; Schmidt, Thomas J.; Bioorganic & Medicinal Chemistry (1999), 7(11), 2343-2352.

55. Fardella, Giuseppe; Chiappini, lone; Ambrogi, Valeria; Grandolini, Giuliano; Archiv der Pharmazie (Weinheim, Germany) (1999), 332(12), 431434.

56. El-Mergawi, R. A.; Sidkey, Mahassen M. A.; Egyptian Journal of Physiological Sciences (1999), Volume Date 1998, 22(2), 239-254.

57. Belyakov, K. V.; Popov, D. M.; Farmatsiya (Moscow) (1999), 48(2), 30-32.

58. Wong, Hector R.; Menendez, Ingrid Y.; Biochemical and Biophysical Research Communications (1999), 262(2), 375-380.

59. Schmidt, T. J.; Pharmaceutical and Pharmacological Letters (1999), 9(1), 9-13.

60. Heinrich, Michael; Schmitz, M. Lienhard; PCT Int. Appl., WO 9933463, 49 pp.

61. Jang, Dae-Sik; Park, Ki-Hun; Lee, Jong-Rok; Ha, Tae-Joung; Park, Yun-Bae; Nam, Sang-Hae; Yang, Min-Suk; Han'guk Nonghwa Hakhoechi (1999), 42(2), 176-179.

62. Berl, Valerie; Lepoittevin, Jean-Pierre; Photochemistry and Photobiology (1999), 69(6), 653-657.

63. Jang, Dae Sik; Park, Ki Hun; Ko, Hack Lyong; Lee, Hyun Sun; Kwon, Byoung Mog; Yang, Min Suk; Saengyak Hakhoechi (1999), 30(1), 74-78.

64. Jang, Dae Sik; Kwon, Byoung Mog; Yang, Min Suk; Saengyak Hakhoechi (1999), 30(1), 70-73.

65. Raddatz, H.; Hillenbrand, B.; Henle, T.; Lebensmittelchemie (1999), 53(3), 60.

66. Hwang, Daniel H.; Fischer, Nikolaus H.; U.S. Pat. No. 5,905,089, 15 pp.

67. Neves, Marta; Morais, Rui; Gafner, Stefan; Stoeckli-Evans, Helen; Hostettmann, K.; Phytochemistry (1999), 50(6), 967-972.

68. Barrero, Alejandro F.; Oltra, J. Enrique; Raslan, Delio S.; Saude, Denia A.; Journal of Natural Products (1999), 62(5), 726-729.

69. De Upton, I. M. Lomniczi; De la Fuente, J. R.; Esteve-Romero, J. S.; Garcia-Alvarez-Coque, M. C.; Carda-Broch, S.; Journal of Liquid Chromatography & Related Technologies (1999), 22(6), 909-921.

70. Paulsen, E.; Andersen, K. E.; Brandao, F. M.; Bruynzeel, D. P.; Ducombs, G.; Frosch, P. J.; Gossens, A.; Lahti, A.; Menne, T.; Shaw, T.; Tosti, A.; Wahlberg, J. E.; Wilkinson, J. D.; Wrangsjo, K.; Contact Dermatitis (1999), 40(2), 72-76.

71. Park, Eun Jung; Kim Jinwoong; Planta Medica (1998), 64(8), 752-754.

72. Todorova, Milka N.; Markova, Miglena M.; Tsankova, Elena T.; Phytochemistry (1998), 49(8), 2429-2432.

73. Elena T.; Taskova, Rilka Ml.; Peev, Dimitar R.; Phytochemistry (1998), 49(8), 2371-2374.

74. Lyss, Guido; Knorre, Alexander; Schmidt, Thomas J.; Pahl, Heike L.; Merfort, Irmgard; Journal of Biological Chemistry (1998), 273(50), 33508-33516.

75. Youssef, Diaa T. A.; Phytochemistry (1998), 49(6), 1733-1737.

76. Willuhn, G.; Skibinski, A.; Schmidt, T. J.; Planta Medica (1998), 64(7), 635-639.

77. Cho, Jae Youl; Park, Jisoo; Yoo, Eun Sook; Baik, Kyong Up; Jung, Jee Hyung; Lee, Jongsoo; Park, Myung Hwan; Planta Medica (1998), 64(7), 594-597.

78. Ruengeler, Peter; Lyss, Guido; Castro, Victor; Mora, Gerardo; Pahl, Heike L.; Merfort, Irmgard; Planta Medica (1998), 64(7), 588-593.

79. Jang, Dae Sik; Park, Ki Hun; Kim, Hwan Mook; Hong, Dong Ho; Chun, Hyo Kon; Kho, Yung Hee; Yang, Min Suk; Saengyak Hakhoechi (1998), 29(3), 243-247.

80. Bruno, Maurizio; Vasallo, Nadia; Fazio, Caterina; Gedris, Thomas E.; Herz, Werner; Biochemical Systematics and Ecology (1998), 26(7), 801-803.

81. Chhabra, B. R.; Gupta, S.; Jain, M.; Kalsi, P. S.; Phytochemistry (1998), 49(3), 801-804.

82. Tan, R. X.; Tang, H. Q.; Hu, J.; Suhai, B.; Phytochemistry (1998), 49(1), 157-161.

83. Pogrebnyak, A. V.; Poroikov, V. V.; Starykh, V. V.; Konovalov, D. A.; Rastitel'nye Resursy (1998), 34(1), 61-64.

84. Enriz, Ricardo D.; Rodriguez, Ana M.; Jauregui, Esteban A.; Acta Farmaceutica Bonaerense (1998), 17(1), 41-52.

85. Ma, Ji-Yuan; Wang, Zheng-Tao; Xu, Luo-Shan; Xu, Guo-Jun; Kadota, Shigetoshi; Namba, Tsuneo; Phytochemistry (1998), 48(1), 201-203.

86. Gurunath, S.; Patewad, B. R.; Sawaikar, D. D.; Panse, G. T.; Pujar, P. P.; Nagasampagi, B. A.; Rojatkar, S. R.; Indian Journal of Chemistry, Section B: Organic Chemistry Including Medicinal Chemistry (1997), 36B(11), 1047-1049.

87. Jang, Dae Sik; Yang, Min Suk; Park, Ki Hun; Planta Medica (1998), 64(3), 289-290.

88. Starykh, V. V.; Konovalov, D. A.; Rastitel'nye Resursy (1997), 33(4), 17-27.

89. Tesevic, Vele; Vajs, Vlatka; Todorovic, Nina; Dokovic, Dejan; Marin, Petar; Milosavljevic, Slobodan; Journal of the Serbian Chemical Society (1998), 63(2), 131-135.

90. Hehner, Steffen P.; Heinrich, Michael; Bork, Peter M.; Vogt, Markus; Ratter, Frank; Lehmann, Volker; Schulze-Osthoff, Klaus; Droge, Wulf; Schmitz, M. Lienhard; Journal of Biological Chemistry (1998), 273(3), 1288-1297.

91. Kim, Dae Keun; Lee, Kang Ro; Zee, Ok Pyo; Phytochemistry (1997), 46(7), 1245-1247.

92. Spring, Otmar; Heil, Norbert; Vogler, Bernhard; Phytochemistry (1997), 46(8), 1369-1373.

93. Buschmann, Holger; Spring, Otmar; Phytochemistry (1997), 46(5), 969-972.

94. Lyss, Guido; Schmidt, Thomas J.; Merfort, Irmgard; Pahl, Heike L.; Biological Chemistry (1997), 378(9), 951-961.

95. Wu, Tian-Shung; Leu, Yann-Lii; Kuoh, Chang-Sheng; Jiang, Shwu-Duan; Chen, Chieh-Fu; Lee, Kuo-Hsiung; Journal of the Chinese Chemical Society (Taipei) (1997), 44(3), 357-359.

96. Bicchi, Carlo; Balbo, Cristina; Rubiolo, Patrizia; Journal of Chromatography, A (1997), 779(1+2), 315-320.

97. Pereira, Paulo Sergio; Dias, Diones Aparecida; Vichnewski, Walter; Nasi, Ana Maria Turco Tucci; Herz, Werner; Phytochemistry (1997), 45(7), 1445-1448.

98. Diaz, Eduardo; Barrios, Hector; Nava, Jose Luis; Enriquez, Raul G.; Guzman, Angel; Leon G., Leticia; Fuentes, Fernando; Fuentes B., Aidee; Quintero, Angelina; Solano, Jose Dolores; Journal of Heterocyclic Chemistry (1997), 34(3), 1037-1041.

99. Seitembetov, T. S.; Izvestiya Natsional'noi Akademii Nauk Respubliki Kazakhstan, Seriya Khimicheskaya (1995), (5), 62-65.

100. De Heluani, Carola S.; de Boggiato, Margarita V.; Catalan, Cesar A. N.; Diaz, Jesus G.; Gedris, Thomas E.; Herz, Werner; Phytochemistry (1997), 45(4), 801-805.

101. Marco, J. Alberto; Sanz-Cervera, Juan F.; Garcia-Lliso, Vicente; Batlle, Natalia; Phytochemistry (1997), 45(4), 755-763.

102. Blanco, Javier G.; Gil, Roberto R.; Alvarez, Cecila I.; Patrito, Luis C.; Genti-Raimondi, Susana; Flury, Alfredo; FEBS Letters (1997), 409(3), 396-400.

103. Mossa, Jaber S.; El-Feraly, Farouk S.; Muhammad, Ilias; Zaw, Kyaw; Mbwambo, Zakaria H.; Pezzuto, John M.; Fong, Harry H. S.; Journal of Natural Products (1997), 60(6), 550-555.

104. Beekman, Aaeron C.; Woerdenbag, Herman J.; van Uden, Wim; Pras, Niesko; Konings, Antonius W. T.; Wikstroem, Hakan V.; Schmidt, Thomas J.; Journal of Natural Products (1997),

105. Merfort, I.; Pietta, P. G.; Mauri, P. L.; Zini, L.; Catalano, G.; Willuhn, G.; Phytochemical Analysis (1997), 8(1), 5-8.

106. Milbrodt, Martina; Schroeder, Frank; Koenig, Wilfried A.; Phytochemistry (1997), 44(3), 471-474.

107. Bork, Peter M.; Schmitz, M. Lienhard; Kuhnt, Michaela; Escher, Claudia; Heinrich, Michael; FEBS Letters (1997), 402(1), 85-90.

108. Kim, Jung H.; Lee, S. J.; Lin, Q.; Bierner, Mark W.; Mabry, Tom J.; Biochemical Systematics and Ecology (1996), 24(7/8), 795-798.

109. Yang, Deuk Suk; Whang, Wan Kyunn; Kim, Ii Hyuk; Archives of Pharmacal Research (1996), 19(6), 507-513.

110. Macias, Francisco A.; Torres, Ascension; Molinillo, Jose M. G.; Varela, Rosa M.; Castellano, Diego; Phytochemistry (1996), 43(6), 1205-1215.

111. Goren, Nezhun; Woerdenbag, Herman J.; Bozok-Johansson, Candan; Planta Medica (1996), 62(5), 419-422.

112. Lin, Yun-Lian; Ou, Jun-Chih; Kuo, Yueh-Hsiung; Lee, Kuo-Hsiung; Lin, Jung-Kuei; Journal of Natural Products (1996), 59(10), 991-993.

113. Hwang, Daniel; Fischer, Nikolaus H.; Jang, Byeong C.; Tak, Heekyung; Kim, Jin K.; Lee, Wan; Biochemical and Biophysical Research Communications (1996), 226(3), 810-818.

114. Montanaro, S.; Bardon, A.; Catalan, C. A. N.; Fitoterapia (1996), 67(2), 185-187.

115. Todorova, Milka N.; Krasteva, Maria L.; Phytochemistry (1996), 42(4), 1231-1233.

116. Hernandez, Luis R.; De Riscala, Elmira C.; Catalan, Cesar A. N.; Diaz, Jesus G.; Herz, Werner; Phytochemistry (1996), 42(3), 681-684.

117. Seitembetov, T. S.; Dzhazin, K. A.; Kulyyasov, A. T.; Adekenov, S. M.; Khimiya Prirodnykh Soedinenii (1995), (2), 325-7.

118. Dzhazin, K. A.; Gatilov, Yu. V.; Adekenov, S. M.; Khimiya Prirodnykh Soedinenii (1995), (1), 79-85.

119. Buketova, G. K.; Turmukhambetov, A. Zh.; Bagryanskaya, I. Yu.; Gatilov, Yu. V.; Adekenov, S. M.; Khimiya Prirodnykh Soedinenii (1995), (1), 69-72.

120. Kisiel, Wanda; Stojakowska, Anna; Malarz, Janusz; Kohlmuenzer, Stanislaw; Phytochemistry (1995), 40(4), 1139-40.

121. Ahmed, Ahmed A.; Spring, O.; El-Razek, Mohamed H. Abd; Hussein, Nadia S.; Mabry, Tom J.; Phytochemistry (1995), 39(5), 1127-31.

122. Kisiel, W.; Barszcz, B.; Phytochemistry (1995), 39(6), 1395-7.

123. Jacobsson, Ulla; Kumar, Vijaya; Saminathan, Shantini; Phytochemistry (1995), 39(4), 839-43.

124. Spring, Otmar; Buschmann, Holger; Vogler, Bernhard; Schilling, Edward E.; Spraul, Manfred; Hoffmann, Manfred; Phytochemistry (1995), 39(3), 609-12.

125. Buschmann, Holger; Spring, Otmar; Phytochemistry (1995), 39(2), 367-71.

126. Willuhn, Guenter; Leven, Walter; Deutsche Apotheker Zeitung (1995), 135(21), 33-6.

127. Abegaz, Berhanu M.; Keige, Annah W.; Diaz, Jesus G.; Herz, Werner; Phytochemistry (1995), 38(6), 1555.

128. Goren, Nezhun; Phytochemistry (1995), 38(5), 1261-4.

129. Gonzalez, Antonio G.; Bermejo, Jaime; Triana, Jorge; Eiroa, Jose Luis; Lopez, Mariana; Journal of Natural Products (1995), 58(3), 432-7.

130. Turdybekov, Koblandy M.; Adekenov, Sergazy M.; Raldugin, Victor A.; Muldakhmetov, Zeinulla M.; Struchkov, Yurii T.; Mendeleev Communications (1995), (2), 42-4.

131. Fernandez, Isabel; Pedro, Jose R.; Polo, Esther; Phytochemistry (1995), 38(3), 655-7.

132. Nowak, Gerard; Acta Societatis Botanicorum Poloniae (1993), 62(1-2), 33-6.

133. Woerdenbag, Herman J.; Merfort, Irmgard; Passreiter, Claus M.; Schmidt, Thomas J.; Willuhn, Guenter; van Uden, Wim; Pras, Niesko; Kampinga, Harm H.; Konings, Antonius W. T.; Planta Medica (1994), 60(5), 434-7.

134. Abegaz, Berhanu M.; Keige, Annah W.; Diaz, Jesus D.; Herz, Werner; Phytochemistry (1994), 37(1), 191-6.

135. Yang, M. H.; Blunden, G.; Patel, A. V.; O'Neill, M. J.; Lewis, J. A.; Planta Medica (1994), 60(4), 390.

136. Akbar, Shahid; (1993) 252 pp. Avail.: Univ. Microfilms Int., Order No. DA9411870 From: Diss. Abstr. Int. B 1994, 54(12, Pt. 1), 6144.

137. Singh, Swaranjit; Goyal, Rita; Gupta, Sanjay K.; Chhabra, B. R.; Kalsi, P. S.; Indian Journal of Chemistry, Section B: Organic Chemistry Including Medicinal Chemistry (1993), 32B(12), 1229-33.

138. Groenewegen, W. A.; Knight, D. W.; Heptinstall, S.; Journal of Pharmacy and Pharmacology (1986), 38(9), 709-12.

139. Woerdenbag, H. J.; Meijer, C.; Mulder, N. H.; De Vries, E. G. E.; Hendriks, H.; Malingre, T. M.; Planta Medica (1986), (2), 112-14.

140. Gaspar, A. R. M. D.; Potgieter, D. J. J.; Vermeulen, N. M. J.; Biochemical Pharmacology (1986), 35(3), 493-7.

141. Amason, J. T.; Philogene, B. J. R.; Duval, F.; McLachlan, D.; Picman, A. K.; Towers, G. H. N.; Balza, F.; Journal of Natural Products (1985), 48(4), 581-4.

142. Negrette, R. E.; Backhouse, N.; Avendano, S.; San Martin, A.; Plantes Medicinales et Phytotherapie (1984), 18(4), 226-32.

143. Nawrof, Jan; Bloszyk, Elzbieta; Grabarczyk, Halina; Drozdz, Bohdan; Daniewski, Wlodzimierz M.; Holub, Miroslav; Prace Naukowe Instytutu Ochrony Roslin (1983), 25(1), 91-8.

144. Mohana, K.; Susan, T.; Purushothaman, K. K.; Arogya (Manipal, India) (1984), 10(1), 42-6.

145. Johnson, Edward Stewart; Hylands, Peter John; Hylands, Deborah Margaret; Eur. Pat. Appl. 98041, 93 pp.

146. Zinchenko, V. V.; Khvorost, P. P.; Bakai, S. I.; Biryuk, V. A.; Tarusin, A. D.; Kravchina, T. S.; Rastitel'nye Resursy (1983), 19(4), 544-8.

147. Hall, I. H.; Lee, K. H.; Imakura, Y.; Sims, D.; Journal of Pharmaceutical Sciences (1983), 72(9), 1008-11.

148. Picman, A. K.; Picman, J.; Towers, G. H. N.; Contact Dermatitis (1982), 8(5), 294-301.

149. Ito, Kazuo; lida, Toshiyuki; Phytochemistry (Elsevier) (1981), 20(2), 271-3.

150. Borges del Castillo, J.; Manresa Ferrero, M. T.; Rodriguez Luis, F.; Vazquez Bueno, P.; Genoves Leonor, N.; Castilo Arevalo, S.; Journal of Natural Products (1981), 44(3), 348-50.

151. Takeda, Reiji; Ohta, Yoshimoto; Hirose, Yoshio; Chemistry Letters (1980), (11), 1461-4.

152. Matsueda, Sumu; Nagaki, Masahiko; Koreeda, Masato; Yakugaku Zasshi (1980), 100(6), 615-18.

153. Shamyanov, I. D.; Sidyakin, G. P.; Khimiya Prirodnykh Soedinenii (1980), (2), 258.

154. Sham'yanov, I. D.; Mallabaev, A.; Sidyakin, G. P.; Khimiya Prirodnykh Soedinenii (1979), (6), 865.

155. Ito, Kazuo; Sakakibara, Yoshihisa; Haruna, Mitsumasa; Chemistry Letters (1979),

(12), 1473-6.

156. Sohi, A. S.; Tiwari, V. D.; Lonkar, A.; Rangachar, S. K.; Nagasampagi, B. A.; Contact Dermatitis (1979), 5(3), 133-6.

157. Koreeda, Masato; Matsueda, Sumu; Satomi, Takehiro; Hirotsu, Ken; Clardy, Jon; Chemistry Letters (1979), (1), 81-4.

158. Stampf, Jean Luc; Schlewer, Gilbert; Ducombs, Georges; Foussereau, Jean; Benezra, Claude; British Journal of Dermatology (1978), 99(2), 163-9.

159. Hausen, B. M.; Herrmann, H. D.; Willuhn, G.; Contact Dermatitis (1978), 4(1), 3-10.

160. Gonzalez, A. G.; Bermejo, J.; Amaro, J. M.; Massanet, G. M.; Galindo, A.; Cabrera, I.; Canadian Journal of Chemistry (1978), 56(4), 491-4.

161. Gonzalez, A. G.; Bermejo, J.; Cabrera, I.; Galindo, A.; Massanet, G. M.; Anales de Quimica (1968-1979) (1977), 73(1), 86-7.

162. Bohlmann, Ferdinand; Zdero Christa; Phytochemistry (Elsevier) (1977), 16(6), 778-9.

163. Sham'yanov, I. D.; Mallabaev, A.; Rakhmankulov, U.; Sidyakin, G. P.; Khimiya Prirodnykh Soedinenii (1976), (6), 819.

164. Tarasov, V. A.; Abdullaev, N. D.; Kasymov, Sh. Z.; Sidyakin, G. P.; Khimiya Prirodnykh Soedinenii (1976), (5), 667-8.

165. Rubinchik, M. A.; Rybalko, K. S.; Evstratova, R. I.; Konovalova, 0. A.; Rastitel'nye Resursy (1976), 12(2), 170-81.

166. Kisiel, Wanda; Polish Journal of Pharmacology and Pharmacy (1975), 27(4), 461-7.

167. Yamakawa, Koji; Nishitani, Kiyoshi; Tominaga, Tomohiro; Tetrahedron Letters (1975), (33), 2829-32.

168. Mitchell, John C.; Epstein, William L.; Archives of Dermatology (1974), 110(6), 871-3.

169. Kwon, Young Myung; Woo, Won Sick; Woo, Lin Keun; Lee, Min Jai; Han'guk Saenghwa Hakhoechi (1973), 6(2), 85-94.

170. Gonzalez Gonzalez, Antonio; Bermejo Barrera, J.; Massanet, G. M.; Perez, J.; Anales de Quimica (1968-1979) (1973), 69(12), 1333-4.

171. McCahon, Charlotte, B.; Kelsey, R. G.; Sheridan, Richard P.; Shafizadeh, F.; Bulletin of the Torrey Botanical Club (1973), 100(1), 23-8.

172. Irwin, M. A.; Geissman, T. A.; Phytochemistry (Elsevier) (1973), 12(4), 863-9.

173. Konovalova, 0. A.; Rybalko, K. S.; Kabanov, V. S.; Khimiya Prirodnykh Soedinenii (1972), (6), 721-4.

174. Huang, Eng-Shang; Lee, Kuo-Hsiung; Piantadosi, Claude; Geissman, T. A.; Pagano, Joseph S.; Journal of Pharmaceutical Sciences (1972), 61(12), 1960-2.

175. Lee, Kuo-Hsiung; Huang, Eng-Shang; Piantadosi, Claude; Pagano, Joseph S.; Geissman, T. A.; Cancer Research (1971), 31(11), 1649-54.

176. Doskotch, Raymond W.; El-Feraly, Farouk S.; Hufford, Charles D.; Canadian Journal of Chemistry (1971), 49(12), 2103-10.

177. Rodriguez, Eloy; Yoshioka, Hirosuke; Mabry, Tom J.; Phytochemistry (Elsevier) (1971), 10(5), 1145-54.

178. Irwin, Michael A.; Geissman, Theodore A.; Phytochemistry (Elsevier) (1971), 10(3), 637-45.

179. Lee, Kuo-Hsiung; Matsueda, S.; Geissman, Theodore A.; Phytochemistry (Elsevier) (1971), 10(2), 405-10.

180. Yoshioka, Hirosuke; Renold, W.; Fischer, N. H.; Higo, Akio; Mabry, Tom J.; Phytochemistry (Elsevier) (1970), 9(4), 823-32.

What is claimed is:

1. A method for treating hepatitis C virus infection, comprising administering to a subject in need thereof an effective amount of a sesquiterpene lactone having a γ-lactone fused with a 10-membered ring, an 8-membered ring that is further fused with a 4-membered ring, a 7-membered ring that is further fused with a 5-membered ring, or a 6-membered ring that is further fused with a 6-membered ring.

2. The method of claim 1, wherein the y-lactone is substituted with a methylene group.

3. The method of claim 1, wherein the y-lactone is substituted with an alkyl group.

4. The method of claim 1, wherein the sesquiterpene lactone is concurrently administered in combination with a second therapeutic agent, in which the second therapeutic agent is IFNα, Intron A, PEG-INTRON, Roferon A, Pegasys, Infergen A, Wellferon, Omniferon, Interferon Omega, Albuferon-α, Rebif, Rebetron, Symmetrel, an NS2-NS3 autoprotease inhibitor, an NS3 protease inhibitor, an NS3 helicase, an NS4 co-factor inhibitor, an NS5B polymerase inhibitor, an IRES Inhibitor, an inosine monophosphate dehydrogenase inhibitor, an E2 inhibitor, an antifibrotic, a caspase inhibitor, a β-tubulin inhibitor, an anti-HCV IgG, an immunosuppressant, or an immune modulator.

5. A method for treating hepatitis C virus infection, comprising administering to a subject in need thereof an effective amount of a sesquiterpene lactone of the formula: 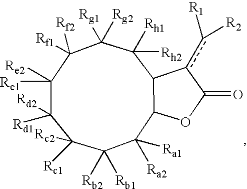
wherein
each of R₁ and R₂, independently, is H, alkyl, cycloalkyl, heterocycloalkyl, aryl, heteroaryl, aralkyl, an amino acid moiety, a polypeptide moiety, F, Cl, Br, I, OR, SR, NRR′, C(O)R, COOR, or O(C)OR; [symbol]
is a single bond or a double bond;
each of Rₐ₁ and Rₐ₂, independently, is H, alkyl, cycloalkyl, heterocycloalkyl, F, Cl, Br, I, OR, SR, C(O)R, COOR, or O(C)OR; or Rₐ₁ and Rₐ₂, taken together, is a methylene group, a carbonyl group, or an epoxy group; or one of Rₐ₁ and Rₐ₂, together with one of R_{b1} and R_{b2}, is a double bond, —CH₂—, or —O—; or one of Rₐ₁ and Rₐ₂, together with one of R_{d1} and R_{d2}, is a single bond or —O—; or one of Rₐ₁ and Rₐ₂, together with one of Rₑ₁ and Rₑ₂, is a single bond, —O—CR₂—O—, or —O—; or one of Rₐ₁ and Rₐ₂, together with one of R_{f1} and R_{f2}, is a single bond or —O—;
each of R_{b1} and R_{b2}, independently, is H, alkyl, cycloalkyl, heterocycloalkyl, F, Cl, Br, I, OR, SR, C(O)R, COOR, or O(C)OR; or R_{b1} and R_{b2}, taken together, is a methylene group, a carbonyl group, or an epoxy group; or one of R_{b1} and R_{b2}, together with one of R_{c1} and R_{c2}, is a double bond, —CH₂—, or —O—; or one of R_{b1} and R_{b2}, together with one of Rₑ₁ and Rₑ₂, is a single bond, —CRR′-CH₂—, or —O—; or one or R_{b1} and R_{b2}, together with one of R_{f1} and R_{f2}, is a single bond or —O—; or one or R_{b1} and R_{b2}, together with one of R_{g1} and R_{g2}, is a single bond or —O—;
each of R_{c1} and R_{c2}, independently, is H, alkyl, cycloalkyl, heterocycloalkyl, F, Cl, Br, I, OR, SR, C(O)R, COOR, or O(C)OR; or R_{c1} and R_{c2}, taken together, is a methylene group, a carbonyl group, or an epoxy group; or one of R_{c1} and R_{c2}, together with one of R_{d1} and R_{d2}, is a double bond, —CH₂—, or —O—; or one of R_{c1} and R_{c2}, together with one of R_{f1} and R_{f2}, is a single bond or —O—; or one of R_{c1} and R_{c2}, together with one of R_{g1} and R_{g2}, is a single bond or —O—; or one or R_{c1} and R_{c2}, together with one of Rₕ₁ and Rₕ₂, is a single bond or —O—;
each of R_{d1} and R_{d2}, independently, is H, alkyl, cycloalkyl, heterocycloalkyl, F, Cl, Br, I, OR, SR, C(O)R, COOR, or O(C)OR; or R_{d1} and R_{d2}, taken together, is a methylene group, a carbonyl group, or an epoxy group; or one of R_{d1} and R_{d2}, together with one of Rₑ₁ and Rₑ₂, is a double bond, —CH₂—, or —O—; or one of R_{d1} and R_{d2}, together with one of R_{f1} and R_{f2}, is —COO—; or one of R_{d1} and R_{d2}, together with one of R_{g1} and R_{g2}, is a single bond, —CRR′-CH₂—, or —O—; or one of R_{d1} and R_{d2}, together with one of Rₕ₁ and Rₕ₂, is a single bond or —O—;
each of Rₑ₁ and Rₑ₂, independently, is H, alkyl, cycloalkyl, heterocycloalkyl, F, Cl, Br, I, OR, SR, C(O)R, COOR, or O(C)OR; or Rₑ₁ and Rₑ₂, taken together, is a methylene group, a carbonyl group, or an epoxy group; or one of Rₑ₁ and Rₑ₂, together with one of R_{f1} and R_{f2}, is a double bond, —CH₂—, or —O—; or one of Rₑ₁ and Rₑ₂, together with one of Rₕ₁ and Rₕ₂, is a single bond or —O—;
each of R_{f1} and R_{f2}, independently, is H, alkyl, cycloalkyl, heterocycloalkyl, F, Cl, Br, I, OR, SR, C(O)R, COOR, or O(C)OR; or R_{f1} and R_{f2}, taken together, is a methylene group, a carbonyl group, or an epoxy group; or one of R_{f1} and R_{f2}, together with one of R_{g1} and R_{g2}, is a double bond, —CH₂—, or —O—;
each of R_{g1} and R_{g2}, independently, is H, alkyl, cycloalkyl, heterocycloalkyl, F, Cl, Br, I, OR, SR, C(O)R, COOR, or O(C)OR; or R_{g1} and R_{g2}, taken together, is a methylene group, a carbonyl group, or an epoxy group; or one of R_{g1} and R_{g2}, together with one of Rₕ₁ and Rₕ₂, is a double bond, —CH₂—, or —O—; and
each of Rₕ₁ and Rₕ₂, independently, is H, alkyl, cycloalkyl, heterocycloalkyl, F, Cl, Br, I, OR, SR, C(O)R, COOR, or O(C)OR; or Rₕ₁ and Rₕ₂, taken together, is a methylene group, a carbonyl group, or an epoxy group;
in which each of R and R′, independently, is H, hydroxy, aryl, alkyl, cycloalkyl, heterocycloalkyl; or R and R′, taken together, is a cycloalkyl or heterocycloalkyl.

6. The method of claim 5, wherein each of R₁ and R₂ is H and [symbol] is a double bond.

7. The method of claim 6, wherein each of Rₐ₁ and Rₐ₂, independently, is H;
each of R_{b1} and R_{b2}, independently, is H or alkyl; or one of Rₐ₁ and Rₐ₂, together with one of R_{b1} and R_{b2}, is a double bond or —O—; each of R_{c1} and R_{c2}, independently, is H, OR, or O(C)OR; or one of R_{b1} and R_{b2}, together with one of R_{c1} and R_{c2}, is a double bond or —O—;
each of R_{d1} and R_{d2}, independently, is H or OR; or one of R_{c1} and R_{c2}, together with one of R_{d1} and R_{d2}, is —O—; each of Rₑ₁ and Rₑ₂, independently, is H or OR; each of R_{f1} and R_{f2}, independently, is H, alkyl, OR, COOR, or O(C)OR; or R_{f1} and R_{f2}, taken together, is a methylene group; or one of Rₑ₁ and Rₑ₂, together with one of R_{f1} and R_{f2}, is a double bond or —O—; each of R_{g1} and R_{g2}, independently, is H, OR, O(C)OR; or R_{g1} and R_{g2}, taken together, is a carbonyl group; or one of R_{f1} and R_{f2}, together with one of R_{g1} and R_{g2}, is a double bond; and each of Rₕ₁ and Rₕ₂, independently, is H, OR, or O(C)OR.

8. The method of claim 7, wherein the sesquiterpene lactone is

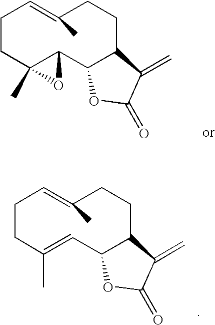

9. The method of claim 6, wherein one of Rₐ₁ and Rₐ₂, together with one of Rₑ₁ and Rₑ₂, is a single bond.

10. The method of claim 9, wherein the other of Rₐ₁ and Rₐ₂ is H or alkyl; each of R_{b1} and R_{b2}, independently, is alkyl; or R_{b1} and R_{b2}, taken together, is a methylene group or a carbonyl group; each of R_{c1} and R_{c2}, independently, is H or OR; or one of R_{b1} and R_{b2}, together with one of R_{c1} and R_{c2}, is a double bond; each of R_{d1} and R_{d2}, independently, is H; or R_{d1} and R_{d2}, taken together, is a carbonyl group; or one of R_{c1} and R_{c2}, together with one of R_{d1} and R_{d2}, is a double bond; the other of Rₑ₁ and Rₑ₂ is H; each of R_{f1} and R_{f2}, independently, is H or alkyl; or R_{f1} and R_{f2}, taken together, is a methylene group; each of R_{g1} and R_{g2}, independently, is H; and each of Rₕ₁ and Rₕ₂, independently, is H or O(C)OR.

11. The method of claim 10, wherein the sesquiterpene lactone is

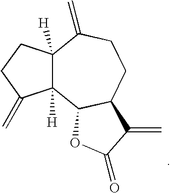

12. The method of claim 6, wherein one of R_{c1} and R_{c2}, together with one of R_{g1} and R_{g2}, is a single bond.

13. The method of claim 12, wherein each of Rₐ₁ and Rₐ₂, independently, is H; each of R_{b1} and R_{b2}, independently, is H or alkyl; the other of R_{c1} and R_{c2} is H; or one of R_{b1} and R_{b2}, together with the other of R_{c1} and R_{c2}, is a double bond; each of R_{d1} and R_{d2}, independently, is H or OR; each of Rₑ₁ and Rₑ₂, independently, is H or OR; or one of R_{d1} and R_{d2}, together with one of Rₑ₁ and Rₑ₂, is a double bond; each of R_{f1} and R_{f1} independently, is H, alkyl, or OR; or R_{f1} and R_{f2} taken together, is a carbonyl group; the other of R_{g1} and R_{g2} is H or alkyl; and each of Rₕ₁ and Rₕ₂, independently, is H, OR, or O(C)OR.

14. The method of claim 13, wherein the sesquiterpene lactone is

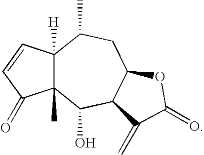

15. The method of claim 6, wherein one of Rₐ₁ and Rₐ₂, together with one of R_{f1} and R_{f2}, is a single bond.

16. The method of claim 15, wherein the other of Rₐ₁ and Rₐ₂ is H; each of R_{b1} and R_{b2}, independently, is H or alkyl; or R_{b1} and R_{b2}, taken together, is a methylene group; each of R_{c1} and R_{c2}, independently, is H; or R_{c1} and R₁₂, taken together, is a carbonyl group; or one of R_{b1} and R_{b2}, together with one of R_{c1} and R_{c2}, is a double bond;
each of R_{d1} and R_{d2}, independently, is H; each of Rₑ₁ and Rₑ₂, independently, is H or OR; or one of R_{d1} and R_{d2}, together with one of Rₑ₁ and Rₑ₂, is a double bond; the other of R_{f1} and R_{f2} is alkyl; each of R_{g1} and R_{g2}, independently, is H; and each of Rₕ₁ and Rₕ₂, independently, is H or O(C)OR.

17. The method of claim 6, wherein one or Rₕ₁ and R_{b2}, together with one of R_{g1} and R_{g2}, is a single bond.

18. The method of claim 17, wherein each of Rₐ₁ and Rₐ₂, independently, is H;
the other of R_{b1} and R_{b2} is alkyl; each of R_{c1} and R_{c2}, independently, is H or OR; each of R_{d1} and R_{d2}, independently, is H; or one of R_{c1} and R_{c2}, together with one of R_{d1} and R_{d2}, is a double bond; each of Rₑ₁ and Rₑ₂, independently, is H; or Rₑ₁ and Rₑ₂, taken together, is a carbonyl group; each of R_{f1} and R_{f2}, independently, is H or alkyl; or R_{f1} and R_{f2}, taken together, is a methylene group; the other of R_{g1} and R_{g2} is H or OR; and each of Rₕ₁ and Rₕ₂, independently, is H; or the other of R_{g1} and R_{g2}, together with one of Rₕ₁ and Rₕ₂, is a double bond.

19. The method of claim 18, wherein the sesquiterpene lactone is

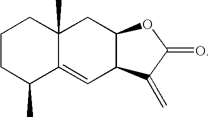

20. The method of claim 5, wherein [symbol] is a single bond.

21. The method of claim 20, wherein each of R₁ and R₂, independently, is H, heterocycloalkyl, SR, or NRR′.

22. The method of claim 21, wherein one of Rₐ₁ and Rₐ₂, together with one of R_{b1} and R_{b2}, is —O—.

23. The method of claim 22, wherein one of Rₑ₁ and Rₑ₂, together with one of R_{f1} and R_{f2}, is a double bond.

24. The method of claim 23, wherein the sesquiterpene lactone is

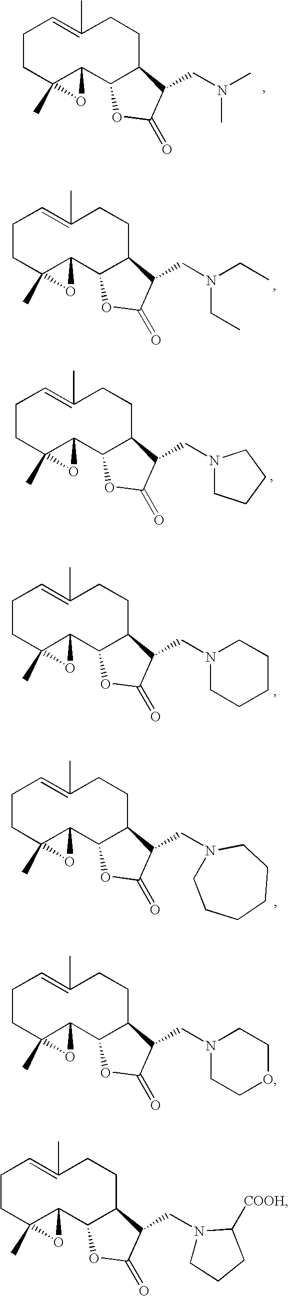

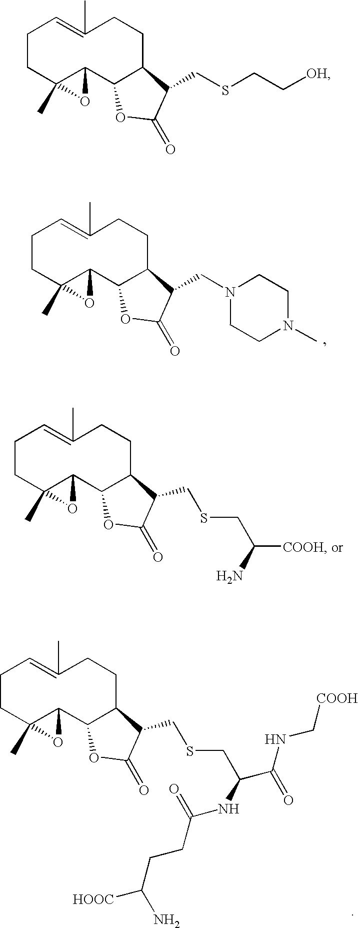

25. The method of claim 20, wherein one of Rₐ₁ and Rₐ₂, together with one of R_{f1} and R_{f2}, is a single bond.

26. The method of claim 25, wherein the other of Rₐ₁ and Rₐ₂, together with one of R_{b1} and R_{b2}, is a double bond or —O—; the other of R_{b1} and R_{b2} is H or alkyl; R_{c1} and R_{c2}, taken together, is a carbonyl group; one of R_{d1} and R_{d2} is H; the other of R_{d1} and R_{d2}, together with one of Rₑ₁ and Rₑ₂, is a double bond or —O—; the other of Rₑ₁ and Rₑ₂ is H; the other of R_{f1} and R_{f2} is alkyl; each of R_{g1} and R_{g2}, independently, is H; and each of Rₕ₁ and Rₕ₂, independently, is H.

27. The method of claim 5, wherein the sesquiterpene lactone is concurrently administered in combination with a second therapeutic agent, in which the second therapeutic agent is IFNα, Intron A, PEG-INTRON, Roferon A, Pegasys, Infergen A, Wellferon, Omniferon, Interferon Omega, Albuferon-α, Rebif, Rebetron, Symmetrel, an NS2-NS3 autoprotease inhibitor, an NS3 protease inhibitor, an NS3 helicase, an NS4 co-factor inhibitor, an NS5B polymerase inhibitor, an IRES Inhibitor, an inosine monophosphate dehydrogenase inhibitor, an E2 inhibitor, an antifibrotic, a caspase inhibitor, a α-tubulin inhibitor, an anti-HCV IgG, an immunosuppressant, or an immune modulator.

28. A sesquiterpene lactone of the formula: 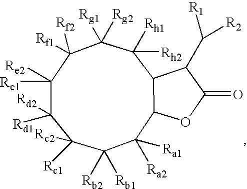
wherein
each of R₁ and R₂, independently, is H, alkyl, cycloalkyl, heterocycloalkyl, aryl, heteroaryl, aralkyl, an amino acid moiety, a polypeptide moiety, F, Cl, Br, I, OR, SR, NRR′, C(O)R, COOR, or O(C)OR;
each of Rₐ₁ and Rₐ₂, independently, is H, alkyl, cycloalkyl, heterocycloalkyl, F, Cl, Br, I, OR, SR, C(O)R, COOR, or O(C)OR; or Rₐ₁ and Rₐ₂, taken together, is a methylene group, a carbonyl group, or an epoxy group; or one of Rₐ₁ and Rₐ₂, together with one of R_{b1} and R_{b2}, is a double bond, —CH₂—, or —O—;
each of R_{b1} and R_{b2}, independently, is H, alkyl, cycloalkyl, heterocycloalkyl, F, Cl, Br, I, OR, SR, C(O)R, COOR, or O(C)OR; or R_{b1} and R_{b2}, taken together, is a methylene group, a carbonyl group, or an epoxy group; or one of R_{b1} and R_{b2}, together with one of R_{c1} and R_{c2}, is a double bond, —CH₂—, or —O—;
each of R_{c1} and R_{c2}, independently, is H, alkyl, cycloalkyl, heterocycloalkyl, F, Cl, Br, I, OR, SR, C(O)R, COOR, or O(C)OR; or R_{c1} and R_{c2}, taken together, is a methylene group, a carbonyl group, or an epoxy group; or one of R_{c1} and R_{c2}, together with one of R_{d1} and R_{d2}, is a double bond, —CH₂—, or —O—;
each of R_{d1} and R_{d2}, independently, is H, alkyl, cycloalkyl, heterocycloalkyl, F, Cl, Br, I, OR, SR, C(O)R, COOR, or O(C)OR; or R_{d1} and R_{d2}, taken together, is a methylene group, a carbonyl group, or an epoxy group; or one of R_{d1} and R_{d2}, together with one of Rₑ₁ and Rₑ₂, is a double bond, —CH₂—, or —O—;
each of Rₑ₁ and Rₑ₂, independently, is H, alkyl, cycloalkyl, heterocycloalkyl, F, Cl, Br, I, OR, SR, C(O)R, COOR, or O(C)OR; or Rₑ₁ and Rₑ₂, taken together, is a methylene group, a carbonyl group, or an epoxy group; or one of Rₑ₁ and Rₑ₂, together with one of R_{f1} and R_{f2}, is a double bond, —CH₂—, or —O—;
each of R_{f1} and R_{f2}, independently, is H, alkyl, cycloalkyl, heterocycloalkyl, F, Cl, Br, I, OR, SR, C(O)R, COOR, or O(C)OR; or R_{f1} and R_{f2}, taken together, is a methylene group, a carbonyl group, or an epoxy group; or one of R_{f1} and R_{f2}, together with one of R_{g1} and R_{g2}, is a double bond, —CH₂—, or —O—;
each of R_{g1} and R_{g2}, independently, is H, alkyl, cycloalkyl, heterocycloalkyl, F, Cl, Br, I, OR, SR, C(O)R, COOR, or O(C)OR; or R_{g1} and R_{g2}, taken together, is a methylene group, a carbonyl group, or an epoxy group; or one of R_{g1} and R_{g2}, together with one of Rₕ₁ and Rₕ₂, is a double bond, —CH₂—, or —O—; and
each of Rₕ₁ and Rₕ₂, independently, is H, alkyl, cycloalkyl, heterocycloalkyl, F, Cl, Br, I, OR, SR, C(O)R, COOR, or O(C)OR; or Rₕ₁ and Rₕ₂, taken together, is a methylene group, a carbonyl group, or an epoxy group;
in which each of R and R′, independently, is H, hydroxy, aryl, alkyl, cycloalkyl, heterocycloalkyl; or R and R′, taken together, is a cycloalkyl or heterocycloalkyl.

29. The sesquiterpene lactone of claim 28, wherein each of R₁ and R₂, independently, is H, heterocycloalkyl, SR, or NRR′.

30. The sesquiterpene lactone of claim 29, wherein one of Rₐ₁ and Rₐ₂, together with one of R_{b1} and R_{b2}, is —O—.

31. The sesquiterpene lactone of claim 30, wherein one of Rₑ₁ and Rₑ₂, together with one of R_{f1} and R_{f2}, is a double bond.

32. The sesquiterpene lactone of claim 31, wherein the sesquiterpene lactone 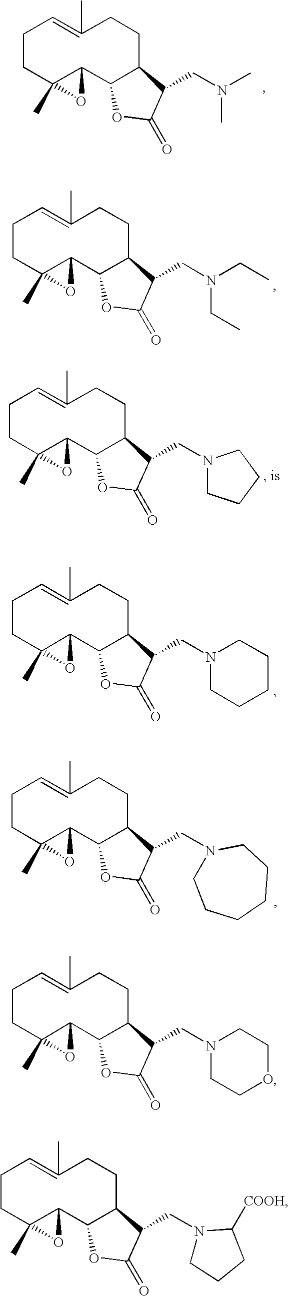 [chemical structure]
